(19) United States
(12) Patent Application Publication
(10) Pub. No.: US 2010/0129794 A1
(43) Pub. Date: May 27, 2010

(54) Magnetic Polymer Particles

(76) Inventors: Roland Fabis, Leverkusen (DE); Sabine Jennrich, Krefeld (DE)

Correspondence Address: LEON R. YANKWICH, 201 BROADWAY, CAMBRIDGE, MA, 02139, US

(21) Appl. No.: 12/085,604

(22) PCT Filed: Dec. 7, 2006
(86) PCT No.: PCT/EP2006/069448

§ 371 Date: Dec. 14, 2009

(30) Foreign Application Priority Data
Dec. 9, 2005 (DE) 10 2005 058 979.0

(51) Int. Cl. G01N 33/545; H01F 1/26; C12Q 1/68

(52) U.S. Cl. 435/6; 252/62.54; 436/531; 435/7.1

(57) ABSTRACT

The present invention relates to magnetic polymer particles including magnetic particles selected from the group of ferromagnetic, ferrimagnetic and/or superparamagnetic particles, where the magnetic particles are embedded in a crosslinked polyacrylate or polyalkylacrylate matrix.

The present invention relates to magnetic polymer particles which include ferromagnetic, ferrimagnetic and/or superparamagnetic particles which are embedded in a crosslinked polyacrylate or poly[(alkylacrylate)]matrix which includes functional groups of the general formula (I)

—C(═O)-M-R (I)

in which

M may be —O—, —NH— or —N(C₁-C₆-alkyl)-;

R may be hydrogen or a group YX in which
Y may be an alkylene group —(CH₂)ₗ— and l may be an integer 1, 2, 3, 4, 5 or 6;
   a hydroxy-substituted alkylene group of the type:
   —[CH₂—CH(OH)—CH₂]_{g} and/or —[CH_{2—}CH(CH₂OH)—]ₕ—
   where g and h may be independently of one another an integer 1, 2, 3, 4, 5 or 6;
   —CH₂—CH₂CH(OH)]— and/or —CH₂—CH₂—CH(OH)—CH₂—CH₂CH(OH)—
   —[CH₂—CH(OH)]ₘ— and/or —[CH(OH)—CH₂]ₙ—
   where m and n may be independently of one another an integer 1, 2, 3, 4, 5 or 6;
   —(CH₂)ₐ—CH(OH)—CH₂-A-(CH2)_{b}-B-C(═O)-[cyclo-C₆H₁₀]—CH₂—,
   where A and B may be independently of one another —NH—, —N(C₁-C₆-alkyl)- or —O— and a may be an integer 1, 2, 3, 4, 5 or 6 and b may be an integer 1, 2, 3, 4, 5, 6, 7 or 8;
X may be hydrogen, —OH, —O—C₁-C₆-alkyl, —O—C₆-C₁₂-aryl, —O—C₇-C₁₄-alkylenearyl with an alkylene chain consisting of 1, 2, 3, 4, 5 or 6 carbon atoms and a C₆-C₁₂-aryl radical;
   —C₁-C₆-alkyl, —C₆-C₁₂-aryl, heteroaryl, an imidazolyl radical which is optionally linked via a C₁-C₆-alkylene group;
   C₇-C₁₄-alkylaryl with an alkylene chain consisting of 1, 2, 3, 4, 5 or 6 carbon atoms and a C₆-C₁₂-aryl radical;
   a substituent of the general formula

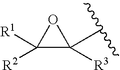

in which
R¹, R² and R³ may be independently of one another hydrogen, C₁-C₆-alkyl and/or C₆-C₁₂-aryl;
—CN, —NC, —N₃;
—C(═O)—R⁴ and R⁴ may be hydrogen, OH, C₁-C₆-alkyl, —O-C₁-C₆-alkyl, C₆-C₁₀-aryl or —O—C₆-C₁₂-aryl;
—NH₂, —NHR⁵ and R⁵ may be hydrogen, C₁-C₆-alkyl and/or C₆-C₁₂-aryl;
F, Cl, Br or I;
—SH or —S—S—H;
2-thiopyridyl or 4-thiopyridyl;
—S(═O)—CH₂—CF₃;
acylimidazole, maleimido or azlactone groups;

or

R may be a group Y′X′L in which
Y′ may be a single bond;
   an alkylene group —(CH₂)_{q}— and q may be an integer 1, 2, 3, 4, 5 or 6;
   a hydroxy-substituted alkylene group of the type:
   —[CH₂—CH(OH)—CH₂]ᵢ— and/or —[CH_{2—}CH(CH₂OH)—]ₒ—
   where i and o may be independently of one another an integer 1, 2, 3, 4, 5 or 6;
   —CH₂—CH₂CH(OH)— and/or —CH₂—CH₂—CH(OH)—CH₂—CH₂CH(OH)—
   —[CH₂—CH(OH)]ᵣ— and/or —[CH(OH)—CH₂]ₛ—
   where r and s may be independently of one another an integer 1, 2, 3, 4, 5 or 6;
   —(CH₂)ₐ—CH(OH)—CH₂-A-(CH2)_{b}-B—C(═O)-[cyclo-C₆H₁₀]—CH₂—,
   where A and B may be independently of one another —NH—, —N(C₁-C₆-alkyl)- or —O— and a may be an integer 1, 2, 3, 4, 5 or 6, and b may be an integer 1, 2, 3, 4, 5, 6, 7 or 8;
   —(CH₂)ₐ—CH(OH)—CH₂-A-(CH2)_{b}-B—C(═O)-[cyclo-C₆H₁₀]—CH₂—,
   in which A and B may be —NH—, a may be an integer 1 or 2, and b may be 6;
X′ may be a single bond;
   —CH(OH)—CH₂—O—, —CH(OH)—CH₂—S—, —CH(OH)—CH₂—NH—, —CH(OH)—CH₂—N(C₁-C₆-alkyl)-, —O—, —C(═O)O—, —C(═O)NH—, —C(═O)N(C₁-C₆-alkyl)-;
   —CR¹R²—R³CH—O—, —O—CR¹R²—CHR³— in which R¹, R² and R³ have the meaning indicated above;
   —NH— or —N(C₁-C₆-alkyl)-;
L may be —C(═O)—NH—(CH₂)ᵤ—[NH—(CH₂)₂]ᵥ—NH₂ and u and v may be independently of one another in each case an integer 1, 2, 3 or 4;
   —(CH₂)_{w}—C(═O)OH and w may be an integer 1, 2, 3, 4, 5 or 6;
   a tri-, tetra- or pentadentate chelating agent such as, for example, a nitrilotriacetic acid residue linked via its ε-N, a so-called low molecular weight, high molecular weight or linear polyethyleneimine residue having a molecular weight of about 500 to 200 000 Da, an amino radical, preferably a polyamine residue, spermidine, cadaverine, diethylenetriamine, spermine, 1,4-bis(3-aminopropyl)piperazine, 1-(2-aminoethyl)piperazine, 1-(2-aminoethyl)piperidine, 1,4,10,13 -tetraoxa-7,16-diazacyclooctadecane, a carboxyl acid residue, or a bound antibody, preferably a secondary antibody, proteins, biotin, oligonucleotides or streptavidin, IDA, DEO or TED (triscarboxymethylethylenediamine) or
   —CH₂—CH₂—N—(CH₂COO⁻)[CH(COO⁻)CH₂COO⁻)].

Preferred magnetic polymers include functional groups of the general formula (I) in which

M may be —O—, —NH— or —N(C₁-C₆-alkyl)-;

R may be hydrogen;
or
a group —YX in which
Y may be an alkylene group —(CH₂)ₗ— and l may be an integer 1, 2, 3, 4, 5 or 6;
   a hydroxy-substituted alkylene group of the type:
   —[CH₂—CH(OH)—CH₂]_{g}— and/or —[CH_{2—}CH(CH₂OH)—]ₕ—
   where g and h may be independently of one another an integer 1, 2, 3 or 4,
   —CH₂—CH₂CH(OH)— and/or —CH₂—CH₂—CH(OH)—CH₂—CH₂CH(OH)—,
   —[CH₂—CH(OH)]ₘ— and/or —[CH(OH)—CH₂]ₙ—
   where m and n may be independently of one another an integer 1, 2, 3 or 4;
   —(CH₂)ₐ—CH(OH)—CH₂-A-(CH2)_{b}-B—C(═O)-[cyclo-C₆H₁₀]—CH₂—,
   in which A and B may be —NH—, a may be an integer 1 or 2, and b may be 6;
X may be hydrogen, —OH, —O—C₁-C₄-alkyl, —O—C₆-C₁₀-aryl, —O—C₇-C₁₄-alkylaryl with an alkylene chain consisting of 1, 2, 3, 4, 5 or 6 carbon atoms and a C₆-C₁₀-aryl radical;
   —C₁—C₆-alkyl, —C₆-C₁₂-aryl, heteroaryl, an imidazolyl radical which is optionally linked via a —C₁-C₆-alkylene group;
   a substituent of the general formula

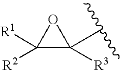

in which
R¹, R² and R³ may be independently of one another hydrogen, C₁-C₃-alkyl;
—NH₂, —NHR⁵ and R⁵ may be hydrogen, C₁-C₄-alkyl;
F, Cl or Br;
—CN, —NC;
—SH or —S—S—H;
2-thiopyridyl or 4-thiopyridyl;
—S(═O)—CH₂—CF₃ (tresyl);
an acylimidazole, maleimido or azlactone group;

or

R may be a group —Y′X′L in which
Y′ may be a single bond;
   an alkylene group —(CH₂)_{q}— and q may be an integer 1, 2, 3, 4, 5 or 6;
   a hydroxy-substituted alkylene group of the type:
   —[CH₂—CH(OH)—CH₂]ᵢ— and/or —[CH_{2—}CH(CH₂OH)—]ₒ—
   where i and o may be independently of one another and integer 1, 2, 3 or 4;
   —CH₂—CH₂CH(OH)— and/or —CH₂—CH₂—CH(OH)—CH₂—CH₂CH(OH)—,
   —[CH₂—CH(OH)]ᵣ— and/or —[CH(OH)—CH₂]ₛ—
   where r and s may be independently of one another an integer 1, 2, 3 or 4;
   —(CH₂)ₐ—CH(OH)—CH₂-A-(CH2)_{b}-B—C(═O)-[cyclo-C₆H₁₀]—CH₂—,
   in which A and B may be —NH—, a may be an integer 1 or 2, and b may be 6;
X′ may be a single bond;
   —CH(OH)—CH₂—O—, —CH(OH)—CH₂—S—, —CH(OH)—CH₂—NH—, —CH(OH)—CH₂—, —N(C₁-C₃ -alkyl)-, —O—, —C(═O)O—, —C(═O)NH—, —C(═O)N(C₁-C₃-alkyl)-;
   —CR¹R²—R³CH—O—, —O—CR¹R²—CHR³— in which R¹, R² and R³ have the meaning indicated above;
   —NH— or —N(C₁-C₃-alkyl)-;
L may be —C(═O)—NH—(CH₂)ᵤ—[NH—(CH₂)₂]ᵥ—NH₂ and u and v may be independently of one another in each case an integer 1, 2, 3 or 4;
   —(CH₂)_{w}—C(═O)OH and w may be an integer 1, 2, 3 or 4;
   a tri-, tetra- or pentadentate chelating agent such as, for example, a nitrilotriacetic acid residue linked via its ε-N, a so-called low molecular weight, high molecular weight or linear polyethyleneimine residue with a molecular weight of preferably 500 to 200 000 Da, a polyamine residue, spermidine, cadaverine, diethylenetriamine, spermine, 1,4-bis(3-aminopropyl)piperazine, 1-(2-aminoethyl)piperazine, 1-(2-aminoethyl)piperidine, 1,4,10,13 -tetraoxa-7,16-diazacyclooctadecane, a carboxyl acid residue, or a bound antibody, preferably a secondary antibody, proteins, biotin, oligonucleotides or streptavidin, IDA, DEO, TED (triscarboxymethylethylenediamine),
   —CH₂—CH₂—N—(CH₂COO^{-)[CH(COO}⁻)CH₂COO⁻)].

Particularly preferred magnetic polymers include the functional groups of the general formula (I) in which

M may be —O— or —NH—;

R may be hydrogen;

or

a group YX in which
Y may be an alkylene group —(CH₂)ₗ—and l may be an integer 1, 2, 3, 4, 5 or 6;
   a hydroxy-substituted alkylene group of the type:
   —[CH₂—CH(OH)—CH₂]_{g}— and/or —[CH_{2—}CH(CH₂OH)—]ₕ—
   where g and h may be independently of one another an integer 1 or 2,
   —CH₂—CH₂CH(OH)— and/or —CH₂—CH₂—CH(OH)—CH₂—CH₂CH(OH)—;
   —[CH₂—CH(OH)]ₘ— and/or —[CH(OH)—CH₂]ₙ—
   where m and n may be independently of one another an integer 1 or 2,
X may be hydrogen, —OH, —O—C₁-C₄-alkyl, —O—C₆-C₁₀-aryl;
   a substituent of the general formula

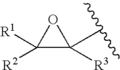

in which R¹, R² and R³ may be independently of one another hydrogen or C₁-C₂-alkyl;
—NH₂;
Cl or Br;
—S(═O)—CH₂—CF₃;

or

R may be a group Y′X′L in which
Y′ may be a single bond;
   an alkylene group —(CH₂)_{q}— and q may be an integer 1, 2 or 3;
   a hydroxy-substituted alkylene group of the type:
   —[CH₂—CH(OH)—CH₂]ᵢ— and/or —[CH_{2—}CH(CH₂OH)—]ₒ—
   where i and o may be independently of one another an integer 1 or 2;
   —[CH₂—CH₂CH(OH)]—,
   —[CH₂—CH(OH)]ᵣ— and/or —[CH(OH)—CH₂]ₛ—
   where r and s may be independently of one another an integer 1 or 2;
X′ may be a single bond;
   —CH(OH)—CH₂—O—, —CH(OH)—CH₂—S—, —CH(OH)—CH₂—NH—, —CH(OH)—CH₂—N(C₁-C₃-alkyl)-, —O—, —C(═O)O—, —C(═O)NH—, —C(═O)N(C₁-C₃-alkyl)-;
   —CR¹R²—R³CH—O—, —O—CR¹R²—CHR³— in which R¹, R² and R³ have the meaning indicated above;
   —NH—;
L may be —C(═O)—NH—(CH₂)₂—[NH—(CH₂)ᵤ]ᵥ—NH₂ in which u may be 1 or 2 and v may be 2;
   —(CH₂)_{w}—C(═O)OH and w may be an integer 1 or 2;
   a tri-, tetra- or pentadentate chelating agent such as, for example, a nitrilotriacetic acid residue linked via its ε-N, a so-called low molecular weight, high molecular weight or linear polyethyleneimine residue with a molecular weight of preferably 500 to 200 000 Da, a polyamine residue, spermidine, cadaverine, diethylenetriamine, spermine, 1,4-bis(3-aminopropyl)piperazine, 1-(2-amino ethyl)piperazine, 1-(2-aminoethyl)piperidine, 1,4,10,13-tetraoxa-7,16-diazacyclooctadecane, a carboxyl acid residue, or a bound antibody, preferably a secondary antibody, proteins, biotin, oligonucleotides or streptavidin, IDA, DEO or DEO or TED (triscarboxymethylethylenediamine),
   —CH₂—CH₂—N—(CH₂COO⁻)[CH(COO⁻)CH₂COO⁻)].

Very particularly preferred magnetic polymers include functional groups of the general formula (I) in which

M may be —O— or —NH—;

R may be hydrogen or
a group YX in which
Y may be a single bond;
   an alkylene group —(CH₂)ₗ— and l may be an integer 1, 2, 3, 4, 5 or 6;
   a hydroxy-substituted alkylene group of the type:
   —[CH₂—CH(OH)—CH₂]_{g}— and/or —[CH_{2—}CH(CH₂OH)—]ₕ—
   where g and h may be independently of one another an integer 1 or 2;
   —[CH₂—CH₂CH(OH)]—
   —[CH₂—CH(OH)]ₘ— and/or —[CH(OH)—CH₂]ₙ—
   where m and n may be independently of one another an integer 1 or 2;
X may be hydrogen;
   a substituent of the general formula

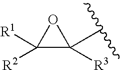

in which
R¹, R² and R³ may be hydrogen;
—NH₂;

or

R may be a group Y′X′L in which
Y′ may be a single bond;
an alkylene group —(CH₂)_{q}— and q may be an integer 1, 2, 3, 4, 5, or 6;
   a hydroxy-substituted alkylene group of the type:
   —[CH₂—CH(OH)—CH₂]ᵢ— and/or —[CH_{2—}CH(CH₂OH)—]ₒ—
   where i and o may be independently of one another an integer 1 or 2;
   —CH₂—CH₂CH(OH)— or
   —[CH₂—CH(OH)]ᵣ— and/or —[CH(OH)—CH₂]ₛ—
   where r and s may be independently of one another an integer 1 or 2;
X′ may be a single bond;
   CH(OH)—CH₂—O—, —CH₂—CH(OH)—O—;
   —CR¹R²—R³CH—O—, —O—CR¹R²—CHR³— in which R¹, R² and R³ have the meaning indicated above;
   —NH—;
L may be —C(═O)—NH—(CH₂)₂—[NH—(CH₂)ᵤ]ᵥ—NH₂ in which u may be 1 or 2, and v may be 2;
   —(CH₂)_{w}—C(═O)OH and w may be an integer 1 or 2;
   a nitrilotriacetic acid residue linked via its ε-N,
   NTA, IDA/DEO, TED, spermine or a secondary antibody
   —CH₂—CH₂—N—(CH₂COO⁻)[CH(COO⁻)CH₂COO⁻)].

The present invention additionally relates to a method for preparing the magnetic polymers and to a method for isolating and/or analyzing at least one biomolecular species from a sample.

In recent years, magnetic particles have been increasingly used in methods for purifying, separating and analyzing various biomolecules. Such magnetic particles ordinarily comprise a magnetic or magnetizable inorganic material which is bound in a glass-like or polymeric matrix. The surface of the magnetic particles is in this case configured so that particular biomolecules from a sample, e.g. a cell lysate, can be bound selectively to this surface. The magnetic particles with the biomolecules bound thereto can easily be removed from the sample by applying an external magnetic field to the sample. The biomolecules can then be eluted by an appropriate treatment of the magnetic particles, and thus be obtained in pure form or in an enriched state.

US patent application 2001/0014468 A1 describes magnetic polymer particles based on polyvinyl alcohol. These particles are prepared by a method in which an aqueous polyvinyl alcohol solution which contains colloidally dispersed magnetic particles is suspended with an organic solution which contains at least two emulsifiers which are immiscible with the polymer phase. The organic solution additionally contains a water-soluble crosslinker by which the polyvinyl alcohol droplets are crosslinked while they are suspended. The magnetic polymer particles obtained in this way can then be activated in accordance with their intended use, or be functionalized by polymeric side chains having suitable functional groups.

French patent application FR 2531452 A1 describes a magnetic carrier matrix which includes a porous refractory metal oxide in whose interior ferromagnetic particles are dispersed. The oxide is impregnated with a crosslinked polyamide with excess bifunctional side groups. This matrix is employed for immobilizing enzymes.

U.S. Pat. No. 4,795,698 describes magnetic polymer particles which are obtained by coprecipitating at least two species of transition metal ions in the presence of a polymer. The polymer contains suitable coordination sites in order to bind the magnetic polymer precipitate. The particles obtained in this way can be employed for immunoassay techniques by selecting suitably functionalized polymers.

U.S. Pat. No. 4,358,388 describes magnetic polymer particles which are prepared by emulsion polymerization of a homogeneous emulsion composed of a dispersion of magnetic particles in an organic polymerizable phase and an aqueous phase which contains at least one emulsifier. The organic phase includes as polymerizable monomers at least one aromatic vinyl compound or a mixture of at least one aromatic vinyl compound and a copolymerizable monomer, for example an alkyl acrylate or an alkyl methacrylate.

The ability of specifically binding one or more species of biomolecules on the surface of magnetic polymer particles is determined to a considerable extent by the nature of the polymer used and the functional groups present on this polymer. Moreover, the requirements to be met by the selectivity of the immobilizing reaction depend on the intended further use of the immobilized biomolecule. It is often sufficient merely to achieve an enrichment of the desired biomolecular species by the immobilization; in other applications, a greater selectivity is necessary or at least desirable in order to obtain the desired biomolecular species in a state which is as pure as possible, directly or with a small number of further purification steps. The magnetic polymer particles should additionally have a binding capacity which is as high as possible for the biomolecular species to be immobilized, in order to enable the use of the magnetic polymer particles to be efficient and cost-effective. However, it is true in principle that a high binding capacity can usually be achieved only with small particle sizes or with a high porosity. However, small particle sizes make it difficult to achieve sufficient magnetizability of the polymer particles.

The magnetic polymers known in the prior art have, however, at most only a very low porosity, or none at all, thus disadvantageously affecting the surface available for the immobilization.

A further problem is that of incorporating the magnetic particles in a suitable polymer matrix. In particular, segregation of the magnetic particles and the organic phase, aggregation of the magnetic particles before or during the polymerization, and achieving an adequate adhesion between polymer matrix and magnetic particles frequently represent problems.

There is thus a need for magnetic polymer particles able to immobilize biomolecules with sufficiently high selectivity. There is in particular a need for magnetic polymer particles which can be functionalized in diverse ways and which can be modified simply, preferably by conventional chemical methods, with ligands able to immobilize biomolecules from a sample with adequate selectivity. It was moreover intended that the modified polymer particles have a maximal binding capacity for the respective biomolecules and, at the same time, have maximal magnetizability.

The object of the present invention is therefore to eliminate or at least alleviate the prior art disadvantages discussed above.

This object is achieved by the magnetic polymer particles as claimed in independent claim 1 and the method as claimed in independent claim 21. Further embodiments, aspects, details and advantages of the present invention are evident from the dependent claims and the following description.

In a first aspect, the present invention relates to magnetic polymer particles, the magnetic polymer particles selected from the group of ferromagnetic, ferrimagnetic and/or superparamagnetic particles. The magnetic particles are embedded in a crosslinked polyacrylate or poly[alkylacrylate]matrix. The polymer matrix includes carboxy groups —C(═O)OH or carboxyl esters or carboxamide groups or other suitable derivatized carboxyl structures as described in the general formula I, each of which may have inter alia a spacer group Y and a reactive group X. The magnetic polymer particles have an average particle size preferably in a range from 5 to 25 μm, particularly preferably in a range from 6 to 20 μm, very particularly preferably in a range from 10 to 15 μm, and pores having a maximum pore radius preferably in a range from 20 to 500 nm, particularly preferably in a range from 30 to 400 nm and very particularly preferably in a range from 80 to 250 nm.

Use of the polyacrylate or poly[(alkyl)acrylate]matrix with their functionalizable carboxyl groups or substituted and functionalizable carboxyl groups makes it possible to prepare relatively large polymer particles which ensure adequate magnetizability, and at the same time make further functionalization with a large number of ligands suitable for immobilizing biomolecules possible in a simple manner.

The polymer matrix initially includes unesterified carboxy groups if free acrylic acid or an (alkyl)acrylic acid such as, for example, methacrylic acid has been used as monomer to be polymerized. If the polymer matrix is to comprise other functionalizable groups, it is possible to employ appropriately derivatized acrylic or (alkyl)acrylic esters—especially methacrylic esters—as monomers, it being possible for the ester residue to be converted in at least a further step into a reactive group which makes possible where appropriate, via a further functionalization, in particular the covalent bonding of in particular ligands which immobilize biomolecules, or spacer groups to which the ligands are eventually linked.

Accordingly, the polymer matrix of the magnetic polymer particles may include functional groups with the general structure —Y—X, where Y is a spacer and X is preferably a reactive group.

It is possible to employ as reactive group X of the functional groups any group which permits a chemical reaction for linking a desired ligand to the spacer. For example, it is possible to use reactive groups X which make substitution reactions on the spacer possible, by which the ligand is covalently bonded to the spacer. Examples of such reactions are etherifications, esterifications, amide formations, the formation of imino linkages and the like.

The reactive groups X of the functional groups are preferably selected from the group consisting of hydrogen, hydroxy, epoxy, aryl, heteroaryl, aralkyl, imidazolyl—where appropriate linked via a C₁ to C₆ alkylene bridge, C(O)H, C(O)R, —C(O)OH, C(O)R, —NH₂, —NHR, azido, cyano, isocyano, —SH, —SSH, thiopyridinyl (2- or 4-thiopyridyl), aryl, halogen (hal), tresyl (2,2,2-trifluoroethanesulfonyl), acylimidazolyl and maleimidolyl or azlactyl groups.

Of the epoxy groups, preference is given to the oxiran-2-yl group, but it is also possible to employ substituted epoxy groups according to the formula shown below:

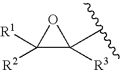

In this case, the radicals R¹, R², and R³ can be selected independently of one another from the group consisting of hydrogen, C₁-C₆-alkyl, C₆-C₁₂-aryl—preferably C₁-C₃-alkyl.

C₁-C₆-Alkyl groups mean in the context of the present invention in particular the following groups: C₁: methyl; C₂: ethyl; C₃: propyl, isopropyl, C₄: butyl, 1-methylpropyl, 2-methylpropyl, 1,1-dimethylethyl, C₅: n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, C₆:hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and/or 1-ethyl-2-methylpropyl. The alkyl groups may optionally be substituted by one or more substituents—such as nitro group(s), amino group(s) and/or one or more halogen atom(s), which may be identical or different. Lower, e.g. —Cᵢ-C₃-alkyl radicals, are defined correspondingly.

Cycloazaalkyl groups mean in the context of the present invention 5- to 16-membered saturated ring systems having 1 to 15—preferably 1 to 12 carbon atom(s) and 1 to 15—preferably 1 to 6, particularly preferably 3 and 4—nitrogen atom(s), which may optionally be substituted by one or more substituents which are selected from the group: lower alkyl radicals having 1 to 6 carbon atom(s) (C₁-C₆-alkyl groups), alkoxy groups having 1 to 6 carbon atom(s) (C₁-C₆-alkoxy groups), nitro groups, amino groups, in turn optionally may be linked via an alkylene group having one, two or three carbon atoms to the cyclic partial structure, and/or one or more halogen atom(s) which may be identical or different. Among these cyclic systems, pyrrolidine, piperidine and piperazine are preferred. The cycloazaalkyl systems may furthermore have, besides nitrogen, also 1, 2, 3 or 4 oxygen atom(s) as ring members, as is the case for example in morpholine.

An aryl radical having 6 to 12 carbon atoms (C₆-C₁₂-aryl) means aromatic substituents which may optionally be substituted by one or more substituents which are selected from the group: lower alkyl radicals having 1 to 6 carbon atom(s) (C₁-C₆-alkyl groups), alkoxy groups having 1 to 6 carbon atom(s) (C₁-C₆-alkoxy groups), nitro groups, amino groups and/or one or more halogen atom(s) which may be identical or different. Embodiments of preferred aryl radicals are for example phenyl, or fused aromatic systems such as naphthyl, or fluorenyl or else systems such as biphenyl. Analogous is true of smaller aryl radicals (e.g. C₆-C₁₀-aryl).

A heteroaryl radical means according to the invention primarily five or six-membered ring systems which have at least one heteroatom. Examples of embodiments are pyrrolyl and furanyl or isothiazolyl on the one hand, and pyridyl, pyrazinyl or triazinyl on the other hand.

An aralkyl radical having 7 to 14 carbon atoms means an aryl radical which is linked via an alkylene bridge. It is moreover possible for the aromatic partial structure and the aromatic partial structure optionally by substituents selected from the group: lower alkyl radicals having 1 to 6 carbon atom(s) (C₁-C₆-alkyl groups), alkoxy groups having 1 to 6 carbon atoms (C₁-C₆-alkoxy groups), nitro groups, amino groups and/or one or more halogen atom(s) which may be identical or different. Aralkyl radicals having six to ten carbon atoms in the aryl radical and one to three carbon atoms in the aliphatic partial structure, such as benzyl or phenethyl, are preferred according to the invention; the benzyl radical is particularly preferred.

Suitable reactive halogen substituents are F, Cl, Br and I, in particular Cl and Br.

Suitable spacers Y can be selected on the basis of synthetic considerations for providing appropriate polymerizable monomers, or the commercial availability thereof, or else the spacer groups are selected with a view to a functionalization which is to be carried out subsequently with an immobilizing ligand. “Spacer” or “spacer group” means in the context of the present invention all chemical groups which can be linked to the carboxy groups of the polymer matrix and thus put the reactive group which is linked to the spacer and which can react with the ligand to form a bond at a distance from the polymer structure of the matrix. It is possible by using these “spacers” to improve the accessibility of the reactive groups for the ligand.

In one embodiment of the invention, the spacer Y of the functional group is selected from the group consisting of:
1. —(CH₂)ₗ— and l may be an integer 1, 2, 3, 4, 5 or 6;
2. hydroxy-substituted alkylene group of the type:
   —[CH₂—CH(OH)—CH₂]_{g}— and/or —[CH_{2—}CH(CH₂OH)—]ₕ—
   where g and h may be independently of one another an integer 1, 2, 3, 4, 5 or 6;
   —CH₂—CH₂CH(OH)— and/or —CH₂—CH₂—CH(OH)—CH₂—CH₂CH(OH)—
   —[CH₂—CH(OH)]ₘ— and/or —[CH(OH)—CH₂]ₙ—
   where m and n may be independently of one another an integer 1, 2, 3, 4, 5 or 6;
3. alkylidene glycols—such as, for example, polyethylene glycol, polypropylene glycol;
   1. mono-, di- or polysaccharides;
   2. polyethyleneimines;
   3. polyacrylic acids;
   4. —CH₂—CH(OH)—CH₂ or —CH₂—C(CH₂OH)H—
   5. —(CH₂)ₐ—CH(OH)—CH₂-A-(CH2)_{b}-B—C(═O)-[cyclo-C₆H₁₀]—CH₂—, where A and B may be independently of one another an —NH—, —N(C₁-C₆-alkyl)- or —O— group, preferably —NH—, a may be an integer 1, 2, 3, 4, 5 or 6—preferably 1 or 2—and b may be an integer 1, 2, 3, 4, 5, 6, 7 or 8—preferably 6.
   6. bifunctional and/or trifunctional crosslinkers (e.g. bifunctional spacers with the following functional groups: imidoester R—C(═NH)—OR′, hydrazides, maleimide, aldehyde, epoxides, iodoacetate, iodoacetamide, acylimidazole, diazoniumaryl, halides, and photoreactive bifunctional spacers such as BASED (bis[b-(4-azidosalicylamido)ethyl]disulfide);
   7. peptide spacers.

The use of a —CH₂— spacer group (Y) in connection with epoxy-functionalized (X) polymer matrices is particularly advantageous because the carboxy group of the acrylic acid can be reacted in a particularly simple manner with epichlorohydrin or epibromohydrin. In a similar manner, it is advantageous to use the spacers mentioned above under item 7) if the carboxy group of the acrylic acid monomer is esterified in a first step with epichlorohydrin or epibromohydrin, and then a reactive group X or a group which comprises a reactive group X is introduced via the epoxy group.

In a further aspect, the present invention relates to magnetic polymer particles which include ferromagnetic, ferrimagnetic or superparamagnetic particles which are embedded in a crosslinked polyacrylate or poly(alkyl)acrylate matrix. In this case, the polyacrylate or poly[(alkyl)acrylate]matrix includes groups of the type R═Y′X′L.

The magnetic polymer particles according to this aspect can be obtained in a simple manner by functionalization of the magnetic polymer particles according to the first aspect described above of the present invention. Accordingly, the statements made in relation to the magnetic polymer particles according to the first aspect also apply analogously to the magnetic polymer particles according to the second aspect of the invention. Differences in relation to the magnetic polymer particles accordingly emerge in respect of the additional functionalization, described in the second aspect, with the appropriate ligands—the structural feature —Y′—X′-L represented for R in the general formula.

In one embodiment of this aspect of the invention, the linking groups X′ by means of which the ligands are linked to the polyacrylate or polyalkylacrylate matrix are selected from the group consisting of —CH(OH)—CH₂—O—, —CH(OH)—CH₂—S—, —CH(OH)—CH₂—NH—, —CH(OH)—CH₂—NR—, —O—, —NH—, —NR—, —C(═O)O—, —C(═O)NH—, —C(═O)NR, and where R is a C₁-C₃-alkyl group. The ligands can thus be linked via imino groups (—NH—), amido groups (—C(═O)NH—), carboxy groups (—C(═O)O—), oxo groups (—O—) or thio groups (—S—) to the polymer matrix. In the case where the ligands are directly linked to the free carboxy groups of the polyacrylate or poly(alkyl)acrylate matrix, the covalent bonding takes place by esterification, amide formation or other derivatization of the carboxy group known from the state of the art. The use of spacers —Y with reactive groups —X to which the ligands can be covalently bonded further extends the range of possible linkage types compared with direct linkage to the carboxy groups, and may improve the accessibility of the reactive group for the ligand—e.g. through the elimination of steric effects.

Accordingly, in one embodiment of the present invention, the ligands able to immobilize biomolecules are directly linked to the carboxy groups of the polymer matrix. Alternatively, they are indirectly linked via spacers to the carboxy groups of the polymer matrix, with the spacers having at least one of the reactive groups described above.

In a further embodiment of this aspect of the present invention, the radicals used as spacers Y to which the ligands can be linked via the appropriate reactive group X are selected from the group consisting of:
a) —(CH₂)ₗ— with l=an integer 1, 2, 3, 4, 5, or 6;
b) hydroxy-substituted alkylene group of the type:
   —[CH₂—CH(OH)—CH₂]_{g}— and/or —[CH_{2—}CH(CH₂OH)—]ₕ—
   where g and h may be independently of one another an integer 1, 2, 3, 4, 5 or 6;
   CH₂—CH₂CH(OH) and/or CH₂—CH₂—CH(OH)—CH₂—CH₂CH(OH)]—
   —[CH₂—CH(OH)]ₘ— and/or —[CH(OH)—CH₂]ₙ—
   where m and n may be independently of one another an integer 1, 2, 3, 4, 5 or 6;
c) alkylidene glycols—such as, for example, polyethylene glycol, polypropylene glycol;
d) mono-, di- or polysaccharides;
e) polyethyleneimines;
f) polyacrylic acids;
g) —(CH₂)ₐ—CH(OH)—CH₂-A-(CH₂)_{b}-B—C(═O)-[cyclo-C₆H₁₀]—CH₂—, where A and B is independently of one another an —NH—, —N(C₁-C₆-alkyl)- or —O— group, preferably —NH—, n is 1-6, preferably 1 or 2, and m is 1-8, preferably 6.
h) bifunctional and/or trifunctional crosslinkers (e.g. bifunctional spacers with the following functional groups: imidoester R—C(═NH)—OR′, hydrazides, maleimide, aldehyde, epoxides, iodoacetate, iodoacetamide, acylimidazole, diazoniumaryl, halides, and photoreactive bifunctional spacers such as BASED (bis[b-(4-azidosalicylamido)ethyl]disulfide);
i) peptide spacers.

These spacers can be either directly linked to the carboxy groups of the matrix, or be linked likewise via further spacers, especially those described in connection with the first aspect of the invention, by means of the reactive groups linked to the spacers, to the carboxy groups.

The ligands able to immobilize preferably biomolecules may be in particular ligands able to immobilize proteins, nucleic acids, oligonucleotides or primary or secondary antibodies. It is possible in particular to use as ligands tri-, tetra- or pentadentate chelating agents, preferably nitrilotriacetic acid residues. Polyethyleneimine residues can also be used. These may be branched or unbranched. A further possibility is to use radicals containing amino groups, for example alkylamino groups of the type —(CH₂)ₙ—NR¹⁰R²⁰, where n, besides 0, is an integer, preferably 1, 2, 3, 4, 5 or 6, in particular 2, 3, 4, 5 or 6, and R¹⁰ and R²⁰ are independently of one another selected from the group consisting of —H and C₁-C₆-alkyl, in particular C₁-C₃-alkyl, amine and/or polyamine residues. A further possibility is to use carboxylic acid residues, proteins, biotin, oligonucleotides, streptavidin or bound antibodies. The polyamines are preferably selected from the group consisting of open-chain and cyclic polyamines having 2, 3, 4, 5 or 6 amino groups. The polyamines may preferably be selected from the group consisting of ethylenediamine, trimethylenediamine, tetramethylenediamine, spermidine, cadaverine, diethylenetriamine, spermine, triethylenetetramine, tetra-ethylenepentamine, pentaethylenehexamine, 1,4-bis(3-aminopropyl)piperazine, 1-(2-aminoethyl)piperazine, 1-(2-aminoethyl)piperidine, 1,4,10,13-tetraoxa-7,16-diazacyclooctadecane and tris(2-aminoethyl)amine and the like.

Carboxylic acid residues which can be employed are in particular carboxy radicals (—C(═O)OH) per se, alkylenecarboxy radicals (-alkylene-C(═O)OH), where the alkyl bridge may be a branched or unbranched C₁-C₁₂-alkyl group, preferably an optionally branched or unbranched C₂-C₆-alkyl group; it is additionally possible to employ carboxy groups linked to aryl partial structures (aryl-C(═O)OH), for example phenylcarboxy groups, i.e. in this case the abovementioned alkylene bridge is replaced by a phenyl system.

There is preferably use in the context of the present invention of ferro- or ferrimagnetic particles, preferably selected from the group consisting of: γ-Fe₂O₃ (maghemite), Cr₂O₃, and ferrites, in particular of the type (M²⁺O)Fe₂O₃, where the M²⁺ is a divalent transition metal cation, and are preferably Fe₃O₄ (magnetite). However, it is likewise possible to use other ferro- or ferrimagnetic particles. These particles have an average particle diameter of less than 5 μm, preferably less than 1 μm, particularly preferably in a range between 0.05 and 0.8 μm, very particularly preferably in a range between 0.1 and 0.4 μm.

Examples of suitable commercially available ferro- or ferrimagnetic particles are ferromagnetic particles based on γ-Fe₂O₃ such as Bayoxide E AB 21 (Lanxess AG, Leverkusen, Germany), ferrimagnetic magnetite obtainable from Lanxess AG, Leverkusen, Germany, as Bayoxide E 8706, E 8707, E 8710 and E 8713H type, and from BASF AG, Ludwigshafen, Germany, as magnetic pigment 340 and magnetic pigment 345.

It is additionally possible also to use superparamagnetic particles. Suitable superparamagnetic materials are Fe, Fe₃O₄, Fe₂O₃, superparamagnetic ferrites, Co, N and binary and/or ternary compounds (alloys). Mention may be made here by way of example of iron oxide crystals with a diameter of about 300 Å or less.

The polymer matrix is crosslinked in the context of the present invention preferably by using di- or polyacrylates or di- or poly(alkyl)acrylates. These are preferably selected from the group consisting of ethylene glycol acrylate, ethylene glycol (alkyl)acrylates, in particular ethylene glycol methacrylate, polyethylene glycol acrylates, polyethylene glycol (alkyl)acrylates, in particular polyethylene glycol methacrylates, pentaerythritol tetraacrylate and pentaerythritol triacrylate, glycerol triacrylate, glycerol trimethacrylate, glycerol propylate triacrylate, glycerol propylate trimethacrylate or else divinylbenzene and its organically modified derivatives such as, for example, 2-hydroxy-1,4-divinylbenzene. These crosslinkers have proved to be particularly suitable in relation to the acrylate and (alkyl)acrylate monomers used.

In a further aspect, the present invention relates to a method for preparing magnetic polymer particles. This method includes the steps:
a) preparation of a dispersion of magnetic particles in a first organic phase, where the magnetic particles are selected from the group consisting of ferromagnetic, ferrimagnetic or superparamagnetic particles, and where the first organic phase includes
   a.1.) one—or more—acrylate monomer(s) selected from the group consisting of acrylic acid, (alkyl)acrylic acids or acrylates and (alkyl)acrylates which include substituted carboxyl groups of the type —C(═O)—O—Y—X, where Y is a spacer group and X is a reactive group,
   a.2.) at least one crosslinker having two or more acrylate or (alkyl)acrylate groups,
   a.3.) at least one lipophilic free-radical initiator, and
   a.4.) at least one organic pore former,
b) mixing and homogenization of the dispersion in a second organic phase to form an emulsion, where the second organic phase includes
   b1) at least one liquid hydrophobic compound and
   b2) at least one surface-active substance, and
c) free-radical polymerization of the emulsion,

where the magnetic polymer particles obtained in this way have an average particle size preferably in the range from 5 to 25 μm, particularly preferably in the range from 6 to 20 μm, very particularly preferably in the range from 10 to 15 μm, and pores having a maximum pore radius preferably in the range from 20 to 500 nm, particularly preferably in a range from 30 to 400 nm, very particularly preferably in the range from 80 to 250 nm.

Magnetic polymer particles like those already described above can be obtained by this method.

The emulsion is preferably flushed with an inert gas before the free-radical polymerization in order thus to drive out any oxygen present in the mixture. Accordingly, the subsequent polymerization is preferably carried out under a protective gas atmosphere. Particularly suitable as protective gas or inert gas in this connection is nitrogen or argon, with preference for nitrogen because of the lower costs. However, other protective gases, especially further noble gases such as helium or krypton, can also be used.

The magnetic particles are preferably ground and/or deagglomerated before or during the preparation of the dispersion. Agglomeration of the primary magnetic particles is prevented thereby, and the dispersing and the subsequent homogenization of the emulsion is facilitated and improved. Ultrasonic techniques, stirring methods and/or grinding methods, for example in a ball mill, can be used for the grinding or deagglomeration.

The free-radical polymerization is preferably carried out at a temperature of 50° C. or higher, preferably at a temperature between 50° C. and 120° C., preferably between 60° C. and 90° C.

In one embodiment of the method of the invention, the monomer in the first organic phase is a compound according to the general formula (II):

H₂C═CR′—C(═O)—OZ (II)

where R′ is H— or a C₁-C₃-alkyl, Z is hydrogen (—H) or a group of the general formula —Y—X, and Y is a spacer and X is as defined above, e.g. selected from the group comprising —OH, —NH₂, —C(═O)OH, halogen (hal), tresyl, maleimido and epoxy groups. The spacer may for example be a —(CH₂)ₗ— group in which l is an integer 1, 2, 3, 4, 5 or 6—or a —CH₂—CH(OH)—CH₂— group. Suitable spacers in this connection are likewise, besides all the groups defined at the outset, those groups which have been mentioned in connection with the magnetic polymer particles according to the aspects described previously, especially the first aspect of the invention.

In a preferred embodiment, the monomer in the first organic phase is selected from the group consisting of glycidyl methacrylate, 2-hydroxyethyl methacrylate, methacrylic acid and acrylic acid, and acrylic acid derivatives of the general formula (III):

H₂C═CR′C(═O)O—(CH₂)_{c}Z (III)

where R′ may be H or methyl, and c is an integer 1, 2, 3, 4, 5, or 6, and Z is selected for example from the group comprising —OH, —NH₂, —C(═O)OH, hal-, tresyl, maleimido and epoxy groups.

Crosslinkers which can be used in the method of the invention are one or more alkylidene glycol diacrylates or alkylidene glycol (alkyl)acrylates of the general formula (III):

H₂C═CR″C(═O)O—[(C_{d}H_{2d}O)]ₑ(C═O)CR′″═CH₂ (IV)

where in formula (IV) R″ and R′″ are independently of one another H or a C₁-C₃-alkyl, and preferably R″ and R′″ are both H or methyl, d is an integer 1, 2, 3 or 4—preferably 1 or 2—and e is an integer between 1 and 100—preferably an integer 1, 2, 3, 4, 5, 6, 7, 8, 9 or 10, and particularly preferably an integer 1, 2, 3, or 4.

It is possible to use in particular ethylene glycol diacrylate, ethylene glycol dimethacrylate or a,ω-di[meth(acrylate)]-functionalized polyethylene glycols or polypropylene glycols—such as, for example, propylene glycol acrylates, propylene glycol (alkyl)acrylates, in particular propylene glycol methacrylates, polypropylene glycol acrylates, polypropylene glycol (alkyl)acrylates, in particular polypropylene glycol methacrylates or propylene glycol acrylates, propylene glycol (alkyl)acrylates, in particular propylene glycol methacrylates, polypropylene glycol acrylates, polypropylene glycol (alkyl)acrylates, in particular polypropylene glycol methacrylates or a mixture thereof as crosslinkers. A further possibility is to use polyacrylates or poly(alkyl)acrylates having at least two, preferably having three or four, acrylate or (alkyl)acrylate groups, where the alkyl group is preferably selected from the group of C₁-C₃-alkyl groups, and is particularly preferably methyl. For example, pentaerythritol tetraacrylate, pentaerythritol tetramethacrylate, pentaerythritol triacrylate, pentaerythritol trimethacrylate, glycerol triacrylate, glycerol trimethacrylate, glycerol propylate triacrylate, glycerol propylate trimethacrylate or else divinylbenzene and its organically modified derivatives such as, for example, 2-hydroxy-1,4-divinylbenzene or mixtures thereof or mixtures of these compounds with other crosslinkers described above can be used.

Compounds which can be used in particular as organic pore formers are those selected from the group consisting of
a) aliphatic, branched or unbranched alcohols having 4 to 20 C atoms, preferably 4 to 16 C atoms and particularly preferably 4 to 8 C atoms, having one or more hydroxy groups, preferably 1-3 hydroxy groups,
b) alkylidene glycols, in particular ethylene glycol, glycerol, etc.
c) carbohydrates such as glucose, and
d) polymeric compounds whose mass-average molecular mass M_{w} is between 200 and 100 000 g/mol and which are selected from the group consisting of polyalkylidene glycol derivatives, polyethyleneimine, polyvinylpyrrolidone and polystyrene, or mixtures of the compounds mentioned above under a), b) and c).

It is possible to employ in particular pore formers selected from the group consisting of ethylene glycol, polyethylene glycol (Mw: 200-20 000 g/mol), polypropylene glycol (Mw: 200-10 000 g/mol), polyethylene glycol monoalkyl ethers (Mw: 200-5000 g/mol), polyethylene glycol dialkyl ethers (Mw: 200-5000 g/mol), polyethylene glycol monoalkyl esters (Mw: 200-20 000 g/mol), polyethylene glycol dialkyl esters (Mw: 200-5000 g/mol), polyethylene glycol diacid (Mw: 1000-20 000 g/mol), polyethyleneimine (Mw: 200-100 000 g/mol), polyvinylpyrrolidone (Mw: 10 000-40 000 g/mol) and/or polystyrene (Mw: 200-5000 g/mol). The pore formers which can be used in particular in the present invention are ethylene glycol and polyethylene glycol (Mw: 1000-6000 g/mol). Also suitable are amino-functionalized polyethylene glycols which are well known in the state of the art as so-called jeffamines.

Lipophilic free-radical initiators which can be used are in particular azoisobutyronitrile (AIBN), 2,2′-azobis(2-amidinopropane) dihydrochloride, 2,2′-azobis(2,4-dimethylvaleronitrile), and 1,1′-azobis(cyclohexane-1-carbonitrile). However, it is also possible to use other free-radical initiators such as, for example, dibenzoyl peroxide, provided that they are sufficiently lipophilic to be able to be incorporated in the dispersion.

The hydrophobic liquid used to prepare the emulsion should be sufficiently chemically inert for it to have no adverse effect on the free-radical polymerization. The liquid should preferably be essentially immiscible with the first organic phase, so that an emulsion of the dispersion in the first organic phase can be produced in the second organic phase. It is crucial to the choice of the systems that the polymer is insoluble in the system during the polymerization.

The hydrophobic liquid can be selected where appropriate from the group consisting of aliphatic or cyclic alkanes, in particular aliphatic alkanes of the general formula C₂H₂ₙ₊₂, where n is >6, aliphatic and cyclic alkenes, in particular aliphatic alkenes of the general formula C₂H₂ₙ, where n is >6, aromatic compounds, in particular monocyclic, bicyclic or tricyclic compounds which may be substituted by alkyl or alkene groups, in particular toluene, xylene, mineral oils, silicone oils, vegetable oils or paraffin oils, substances based on compounds of fatty acids and alcohols, and mixtures of the aforementioned substances, in particular mixtures of aliphatic alkanes and aromatic compounds.

The surface-active substance in the second organic phase is preferably an emulsifier selected from the group consisting of cationic, anionic and nonionic emulsifiers. It is possible to use as surface-active substances for example sorbitan esters, ethoxylated sorbitan esters, polyoxyethylene alkyl phenol ethers and other commercially available surface-active compounds or compound mixtures. Examples of suitable surface-active substances are commercially available substances such as Tween® 20 (polyoxy-ethylene(20)sorbitan monolaurate), Triton®X 100 (t-octylphenoxypolyethoxyethanol), Span 85® (sorbitan trioleate) (all obtainable from Sigma-Aldrich, Taufkirchen, Germany), Hypermer 2296 (obtainable from Uniqema, Gouda, Holland) and similar substances.

The magnetic particles which can be employed in the method are the same particles as described above in connection with the magnetic particles of the invention.

The abovementioned components are mixed according to the invention in the following ratios of amounts (% by weight, wt %) (based on the reaction mixture:

The crosslinker is preferably introduced in a ratio of 0.1-20 wt %, preferably in a range from 0.5 to 5 wt %, particularly preferably 1-4 wt %.

The functionalized monomer is introduced in a ratio of 0.1-20 wt %, preferably 0.5-5 wt %, particularly preferably 1-4 wt %.

The magnetic material or the magnetite is introduced in a ratio of 0.1-20 wt %, preferably 0.5-5 wt %, particularly preferably 1-4 wt %.

The initiator or free-radical initiator is introduced in a ratio of 0-5 wt %, preferably 0.01-3 wt %, particularly preferably 0.05-0.5 wt %.

The detergent (surfactant) is introduced in a ratio of 0-20 wt %, preferably 0.1-10 wt %, particularly preferably 0.1-3 wt %.

The porogen is introduced in a ratio of 0.1-20 wt %, preferably 0.5-5 wt %, particularly preferably 1-4 wt %.

The resulting polymers suitable for the use according to the invention advantageously have the following composition:

The crosslinker content is between 1-95 wt %, preferably between 10-80 wt %, particularly preferably between 20-70 wt % and very particularly preferably between 15-40 wt %.

The polymers formed from the functionalized monomer(s) according to the invention is present in the magnetic particles in a proportion between 1-99 wt %, preferably between 10-80 wt %, particularly preferably between 20-70 wt % and very particularly preferably between 30-60 wt %.

The content of magnetite or magnetic material is between 1-95 wt %, preferably between 10-80 wt %, particularly preferably between 20-70 wt % and very particularly preferably between 30-60 wt %.

In a further embodiment of the method of the invention, as further step d) a functionalization is carried out on the magnetic polymer particles obtained by the method, in which at least one ligand able to immobilize biomolecules is linked to the polymer matrix. The magnetic particles are preferably isolated and washed after the polymerization before they are reacted further, for example functionalized. The particles can be isolated by simple filtration and then be purified by washing with a solvent. Suitable solvents for the washing are both organic and inorganic solvents such as, for example, toluene, acetone or water.

In a variant of the method of the invention, the ligand is directly linked to the reactive groups of the functional groups of the magnetic polymer particle. As an alternative thereto, the ligand can be linked by, in a first step d1), linking a spacer compound having at least two reactive groups to the functional groups of the magnetic polymer particles and then, in a second step d2), covalently bonding the ligand, which is preferably able to immobilize biomolecules, to the magnetic polymer particles.

Suitable ligands and spacers are for example the groups described above in connection with the magnetic particles of the invention. These groups can be linked by appropriate compounds including at least two reactive groups to the polymer particles.

The magnetic polymer particles of the invention as described above in the first and second aspect of the present invention are obtainable by the method of the invention described above.

In a further aspect, the present invention relates to a method for isolating and/or analyzing at least one species of a biomolecule from a sample, the method comprising the steps:

a) provision of a sample containing at least one biomolecular species,

b) contacting the sample with the magnetic polymer particles of the invention under conditions with which the at least one biomolecular species binds to the magnetic polymer particles, and

c) removing the magnetic polymer particles with the bound biomolecules by use of at least one magnetic field.

In one embodiment of this method, elution of the at least one biomolecular species from the magnetic polymer particles follows as further step d).

The magnetic polymer particles prepared by the method of the invention can thus be used for immobilizing or binding preferably biomolecules. It is possible in this connection for the biomolecular species which is bound to the magnetic particles to be selected from the group consisting of nucleic acids, oligonucleotides, proteins, polypeptides, peptides, carbohydrates, lipids, and combinations thereof. It is possible in particular to bind nucleic acids and oligonucleotides, preferably plasmid DNA, genomic DNA, cDNA, PCR DNA, linear DNA, RNA, ribozymes, aptamers, and chemically synthesized or modified nucleic acids or oligonucleotides to the magnetic particles. “Bound” means in this connection generally that the biomolecule develops such a strong interaction with the magnetic polymer particles that it can be removed together with them from a sample under the influence of a magnetic field. These interactions may vary in nature. For example, the interactions may be based on the formation of covalent bonds and/or hydrogen bonds and/or van-der-Waals forces.

The magnetic polymer particles functionalized with a chelating agent as ligand, such as, for example, Ni-NTA, can be used in particular for protein purification.

The magnetic polymer particles modified with amino group-containing ligands such as polyethyleneimine or with carboxyl acid-containing ligands can be used in particular for the isolation and purification of nucleic acids. If secondary antibodies are bound as ligands to the magnetic polymer particles, these can be employed for isolating and purifying primary antibodies.

The sample comprising the at least one biomolecular species may be relatively complex samples such as, for example, blood, tissue, cells, vegetable materials and the like. Other samples are solutions obtained during the course of a purification, amplification, or analytical method, for example PCR solutions.

Further methods in which the magnetic particles of the invention can be employed are nucleic acid detections by means of hybridization, binding of antibodies or organic macromolecules, and binding and detection of biomolecules or cells. The magnetic particles can generally be used for binding, detection and purification of biomolecules or cells.

The present invention is further described by means of various examples. These examples serve merely to illustrate the invention further and are not to be understood as restrictive.

### EXAMPLE 1

### Synthesis of Porous, Hydroxy-Functionalized Magnetic Polymer Particles

In a first step, 8 ml of Tween 20 (Sigma-Aldrich, Taufkirchen, Germany, Aldrich cat. No. 27,4348) were dissolved in 400 ml of paraffin oil (Sigma-Aldrich, Taufkirchen, Germany, Aldrich cat No. 33,076-0). Then 2 ml of ethylene glycol dimethacrylate (Sigma-Aldrich, Taufkirchen, Germany, Aldrich cat. No. 33,568-1) and 9 ml of hydroxyethyl methacrylate (Sigma-Aldrich, Taufkirchen, Germany, Aldrich cat. No. 47,702-8), which are essentially free of inhibitors, were pipetted into a plastic vessel, preferably a 50 ml Falcon tube (BD Biosciences), and 10 ml of polyethylene glycol, 3400, (Sigma-Aldrich, Taufkirchen, Germany, Aldrich cat. No. 20,244-4), 0.3 g of azobis-2-methylpropionitrile (Sigma-Aldrich, Taufkirchen, Germany, Fluka cat. No. 11630) and 7.5 g of BASF magnetite 345 (BASF AG, Ludwigshafen, Germany) are added. This mixture is then homogenized in a Polytron homogenizer at the highest setting for one minute. Half of the paraffin oil solution is put into a 500 ml Nalgene bottle. The magnetite suspension is then added, and the mixture is homogenized while cooling in ice for 120 seconds. The other half of the paraffin oil solution is put under protective gas into a 1000 ml three-neck flask with reflux condenser and KPG stirrer. The initial stirring speed of 500 rpm is subsequently increased to 600 rpm. The iron oxide suspension is then added. The reaction mixture is then freed of oxygen by flushing protective gas through the flask. The reaction temperature is raised to 70° C. for one hour and then kept at 80° C. overnight. The following day, the mixture is filtered, washed with toluene, acetone and water and dried in a vacuum oven at 50° C. The particles obtained in this way are hydroxy-functionalized, macroporous and have a particle diameter of from 10 to 15 μm.

### EXAMPLE 2

### Synthesis of Porous, Epoxy-Functionalized Magnetic Polymer Particles

In a first step, 8 ml of Span 60 (Sigma-Aldrich, Aldrich cat. No. 31,822-1) are dissolved in 400 ml of paraffin oil (Sigma-Aldrich, Aldrich cat. No. 33,076-0). Then 5 ml of ethylene glycol dimethacrylate and 5 ml of glycidyl methacrylate (Sigma-Aldrich, Aldrich cat. No. 15-123-8), which are essentially free of inhibitors, are pipetted into a Falcon tube, and 10 ml of polyethylene glycol (MW 4600) (Sigma-Aldrich, Aldrich cat. No. 37,300-1), 0.3 g of azobis-2-methylpropionitrile and 7.5 g of Bayer Bayoxide E 8710 (Lanxess AG, Leverkusen, Germany) are added. This mixture is homogenized with a Polytron homogenizer at the highest setting for one minute.

Half of the paraffin oil solution is put into a 500 ml Nalgene bottle. Then the magnetite suspension is added and the mixture is homogenized while cooling in ice for 120 seconds. The other half of the paraffin oil solution is then put under protective gas into a 1000 ml three-neck flask with reflux condenser and KPG stirrer. The initial stirring speed of 500 rpm is subsequently increased to 600 rpm. The iron oxide suspension is then added, and the reaction mixture is freed of oxygen by flushing a protective gas through the flask. The reaction temperature is raised to 60° C. for one hour and then kept at 70° C. overnight. The following day, the mixture is filtered and washed with toluene, acetone and water and dried in a vacuum oven at 50° C. The particles obtained in this way are epoxy-functionalized, macroporous and have a particle diameter of from 10 to 15 μm.

### EXAMPLE 3

### Synthesis of Porous, Carboxy-Functionalized Magnetic Polymer Particles

In a first step, 8 ml of Span 60 are dissolved in 400 ml of paraffin oil (Sigma-Aldrich, Aldrich cat. No. 33,076-0). Then 3 ml of ethylene glycol dimethacrylate and 7 ml of methacrylic acid (Sigma-Aldrich, Aldrich cat. No. 39,537-4), which are essentially free of inhibitors, are pipetted into a Falcon tube, and 10 ml of polyethylene glycol, MW 2000 (Sigma-Aldrich, cat. No. 29,590-6), 0.3 g of azobis-2-methylpropionitrile and 7.5 g of Bayer Bayoxide E 8713 H are added. This mixture is homogenized in a Polytron homogenizer at the highest setting for one minute. Half of the paraffin oil solution is put into a 500 ml Nalgene bottle. The magnetite suspension is then added, and the mixture is homogenized while cooling in ice for 120 seconds. The other half of the paraffin oil solution is then put under protective gas into a 1000 ml three-neck flask with reflux condenser with KPG stirrer. The initial stirring speed of 500 rpm is raised to 600 rpm. The iron oxide suspension is added. The reaction mixture is then freed of oxygen by flushing the flask with a protective gas. The reaction temperature is raised to 70° C. for one hour and then kept at 80° C. overnight. The following day, the mixture is filtered, washed with toluene, acetone and water and dried in a vacuum oven at 50° C. The particles obtained in this way are carboxy-functionalized, macroporous and have a particle diameter of from 10 to 15 μm.

### EXAMPLE 4

### Chemical Modification of Porous Magnetic Polymer Particles with Ni-nitrilotriacetic Acid

5 g of the porous magnetic polymer particles from example 1 are suspended in 100 ml of 0.5 M sodium hydroxide solution in a 250 ml round-bottomed flask. Then 2 ml of epibromohydrin are added, and the mixture is left to react in a Rotavapor at 40° C. for 4 hours. The suspension is then filtered through a glass suction filtration apparatus and washed six times with deionized water. The residue is then transferred into a 250 ml round-bottomed flask, suspended in 100 ml of a 0.5 M solution of a-N,N′-bis(carboxymethyl)lysine (synthesis as disclosed by Doebeli et al., EP 0 253 303) and heated in a Rotavapor overnight. The mixture is then filtered with suction and washed four times with deionized water. The magnetic particles are then suspended in 50 ml of a 2% strength nickel sulfate solution and stirred for a further three hours. Subsequently, with magnetic removal, the particles are washed three times with water, resuspended and stored in 50 ml of a 100 mM acetate buffer, pH 6.0, with 20% ethanol.

### EXAMPLE 5

### Chemical Modification of Porous Magnetic Polymer Particles with Ni-nitrilotriacetic Acid

4 g of the polymer particles from example 2 are suspended in 50 ml of deionized water in a 250 ml round-bottomed flask. Then 2 g of the a-N,N′-bis(carboxymethyl)lysine ligand (synthesis described in Doebeli et al., EP 0 253 303) are added, and the reaction mixture is heated at 60° C. while stirring slowly for ten hours. The mixture is then washed with deionized water four times, with magnetic removal. The magnetic particles are then suspended in 50 ml of a 2% strength nickel sulfate solution and stirred for a further three hours. Subsequently, with magnetic removal, the particles are washed three times with water, resuspended and stored in 50 ml of a 100 mM acetate buffer, pH 6.0, with 20% ethanol.

### EXAMPLE 6

### Chemical Modification of Porous Magnetic Polymer Particles with Polyethyleneimine

4 g of the polymer particles from example 3 are suspended in 50 ml of a 10% strength solution of high molecular weight polyethyleneimine (Sigma-Aldrich, Aldrich cat. No. 40,872-7) in water, pH 10, and transferred into a round-bottomed flask. Then 200 mg of N-hydroxysulfosuccinimide sodium salt (Sigma-Aldrich, Fluka cat. No. 56485) and 200 mg of N-3-dimethylamino-N′-propylcarbodiimide hydrochloride (Sigma-Aldrich, Fluka cat. No. 03449) are added, and the mixture is stirred in a Rotavapor at room temperature for three hours. The mixture is then washed six times with deionized water with magnetic removal and is finally suspended in 50 ml of deionized water.

### EXAMPLE 7

### Chemical Modification of Porous Magnetic Polymer Particles with Polyethyleneimine

4 g of the polymer particles from example 2 are suspended in 50 ml of a 10% strength solution of high molecular weight polyethyleneimine (Sigma-Aldrich, Aldrich cat. No. 40,872-7) in water, pH 10, and transferred into a round-bottomed flask and heated at 60° C. while stirring for ten hours. The mixture is then washed six times with deionized water with magnetic removal, and finally suspended in 50 ml of deionized water.

### EXAMPLE 8

### Chemical Modification of Porous Magnetic Polymer Particles with Amines

4 g of the polymer particles from example 3 are suspended in 50 ml of a 5% strength spermine solution (Sigma-Aldrich, Fluka cat. No. 85590), pH 10, and transferred into a round-bottomed flask. Then 200 mg of N-hydroxysulfosuccinimide sodium salt (Sigma-Aldrich, see above) and 200 mg of N-3-dimethylamino-N′-propyl-carbodiimide hydrochloride (Sigma-Aldrich, see above) are added, and the mixture is stirred in a Rotavapor at room temperature for three hours. The mixture is then washed six times with deionized water with magnetic removal, and finally suspended in 50 ml of deionized water.

### EXAMPLE 9

### Chemical Modification of Porous Magnetic Polymer Particles with Amines

4 g of the polymer particles from example 2 are transferred in 50 ml of a 5% strength spermine solution (Sigma-Aldrich, see above), pH 10, into a round-bottomed flask and stirred at 60° C. with stirring for ten hours. The mixture is then washed six times with deionized water with magnetic removal, and finally suspended in 50 ml of deionized water.

### EXAMPLE 10

### Chemical Synthesis of Porous, Carboxy-Functionalized Magnetic Polymer Particles

4 g of the polymer particles from example 2 are suspended in 50 ml of a 10% strength 6-aminohexanoic acid solution (Sigma-Aldrich, Aldrich cat. No. A4,460-6), pH 9.5, transferred into a round-bottomed flask and heated at 60° C. with stirring for ten hours. The mixture is then washed six times with deionized water with magnetic removal, and finally suspended in 50 ml of deionized water.

### EXAMPLE 11

### Binding of Secondary Antibodies to the Porous Magnetic Polymer Particles

2 g of the magnetic polymer particles from example 2 are added to 50 ml of a 10% strength solution of 1,6-diaminohexane (Sigma-Aldrich, Aldrich cat. No. H1-1,169-6) in 100 mM sodium chloride, pH 9.5, and transferred into a 100 ml round-bottomed flask. In a next step, 250 mg of N-hydroxysulfosuccinimide and 250 mg of N-3-dimethylamino-N′-propylcarbodiimide hydrochloride are added. The flask is then transferred onto a Rotavapor and the reaction mixture is left to react at room temperature for three hours.

After cooling, the polymer suspension is washed with deionized water three times with magnetic removal. The beads are suspended in 4 ml of phosphate-buffered saline and then 1 ml of a 10 mg/ml solution of sulfo-SMCC (obtainable from Pierce, Rockford, Ill., USA) in phosphate-buffered saline is added. The suspension is immediately vortexed and then left to react in an end-over-end shaker for two hours. The reaction product is removed from the supernatant by magnetic removal and washed twice with 100 mM phosphate buffer, pH 7.0. Then 1 ml of an antibody solution (1 mg/mg goat anti-mouse IgG; Sigma-Aldrich, Sigma cat. No. M 8642) and 1 ml of phosphate-buffered saline are added, and the reaction mixture is left to react in an end-over-end shaker for two hours. The supernatant is then removed from the product by magnetic removal. The magnetic particles are washed three times with phosphate-buffered saline and can be stored at −20° C.

### EXAMPLE 12

### Binding of Secondary Antibodies to Porous Magnetic Polymer Particles

2 g of the magnetic polymer particles from example 3 are added to 50 ml of a 10% strength solution of 1,6-diaminohexane (Sigma-Aldrich, see above) in 100 mM sodium chloride solution, pH 9.5, and transferred into a 100 ml round-bottomed flask. The flask is then transferred onto a Rotavapor and the reaction mixture is left to react at 70° C. for ten hours. After cooling, the polymer suspension is washed three times with deionized water with magnetic removal. The beads are then suspended in 4 ml of phosphate-buffered saline, and 1 ml of a 10 mg/ml solution of sulfo-SMCC in phosphate-buffered saline is added. The suspension is immediately vortexed and is then left to react in an end-over-end shaker for two hours. The reaction product is separated from the supernatant by magnetic removal and washed twice with 100 mM phosphate buffer, pH 7.0. Then 1 ml of an antibody solution (1 mg/mg, sec. goat anti-mouse) and 1 ml of buffered saline are added, and the mixture is left to react in an end-over-end shaker for two hours. The supernatant is then removed from the product by a magnetic deposition. The magnetic particles are washed three times with phosphate-buffered saline and can be stored at −20° C.

### EXAMPLE 13

### Protein Purification with Ni-NTA Modified, Porous Magnetic Polymer Particles (Denaturing Conditions)

5 ml of cell culture pellets (plasmid pQE 16 in E. coli, transformation and production of recombinant proteins are described in “The Qiaexpressionist”, 3^{rd} edition, Qiagen GmbH, Hilden, 1997) are resuspended in 1 ml of lysis buffer (6M guanidine HCl, 0.1 M NaH₂PO₄, 0.01 M tris×HCl, 0.05% Tween® 20, pH 8.0) by pipetting up and down and shaking the tube at room temperature for one hour. The lysate is then clarified by centrifugation at 10 000 g for 30 minutes, and the supernatant is transferred into another tube. 5 mg of the porous magnetic polymer beads from example 4 or 5 are put into a second tube, and 500 μl of the clarified lysate solution are added. The suspension is incubated on an end-over-end shaker at room temperature for 30 minutes. The tube is then placed on a suitable magnetic separator, and the supernatant is removed by pipetting. In the next step, 500 μl of washing buffer (8 M urea, 0.1 M NaH₂PO₄, 0.01 M tris×HCl, 0.05% Tween® 20, pH 6.3, are added), the tube is placed on a magnetic separator for one minute, and the supernatant is removed by pipetting. This washing step is repeated once with washing buffer. Then 100 μl of elution buffer (8 M urea, 0.1 M NaH₂PO₄, 0.01 M tris×HCl, 0.05% Tween® 20, pH 4.5) are added, the suspension is incubated on an end-over-end shaker for one minute, the tube is placed on a magnetic separator for one minute, and the eluate is collected. The elution step is repeated and the eluates are collected. The protein-binding capacity determined by the Bradford assay is approximately 10 μg/g of magnetic particles.

### EXAMPLE 14

### Concentration of Viruses Using Polyethyleneimine-Modified, Porous Magnetic Polymer Beads

1 ml of virus plasma is put into a 2 ml Eppendorff tube. Then 200 μl of a suspension of polyethyleneimine-modified particles from example 5 are added in a concentration of 10 mg/ml in 200 μl of RNase-free water. The mixture is vortexed, subsequently incubated at room temperature for 15 minutes and then centrifuged at 2000 rpm for two seconds in order to remove residues of particles at the upper end of the tubes. The particles are then magnetically removed over the course of five minutes. The supernatant is discarded without removing particles at the same time. Subsequently, 13.2 ml of the “AL” buffer (Qiagen GmbH, Hilden, Germany, cat. No. 19075) with 82.9 μl of quantification standard (for Cobas TaqMan, Roche) and carrier RNA (in QIAamp® MinElute Vacuum Kit, Qiagen, Hilden, Germany, cat. No. 57714) are mixed with 75 μl of protease solution (in QIAamp® MinElute Kit) and 7 ml of resuspension buffer (in QIAamp® MinElute Kit).

15 μl of enzyme solution (Smitest solution I, JSR Corporation, Ibaraki, Japan), 380 μl of sample diluent (Smitest solution II), and 5 μl of precipitant (Smitest solution IV) are added. Immediately after the addition of the Smitest solutions, the sample is mixed by vortexing. It is then incubated at 55° C. for 30 minutes, followed by a brief centrifugation step at 2000 rpm. The particles are resuspended by repeated pipetting up and down. The suspension of the particles is put onto an Ultrafree MC filter (Millipore Corporation), the tube is capped and the hinge region is cut through with scissors. A filtration device is then put into a 2 ml Eppendorff tube. It is centrifuged at 10 000 rpm for three minutes. The filtration device is removed and discarded. Then 250 μl of a Smitest solution III (for protein solution) which contains quality standard from the RT and cRNA (carrier RNA/Roche Diagnostics) are added. The mixture is immediately vortexed and incubated at 55° C. for 15 minutes. Then 600 μl of isopropanol are added and mixed by inverting the tube 20 times. It is then incubated on ice for 15 minutes and subsequently centrifuged at 15 000 rpm and 4° C. for 10 minutes. The supernatant is cautiously removed without resuspending the particles. 500 μl of 70% ethanol are added and mixed by inverting the tube three times. It is centrifuged at 12 000 rpm and 4° C. for three minutes and then 500 μl of 70% ethanol are added and again mixed by inverting the tube three times. Centrifugation is again carried out at 12 000 rpm and 4° C. for three minutes. The supernatant is removed and discarded. The supernatant after centrifugation at 15 000 rpm for ten seconds is removed. The particles are dried at room temperature for ten minutes. The particles are resuspended in 60 μl of RNase-free water and shaken with a thermomixer at room temperature for 10 to 15 minutes. 50 μl of the eluate are combined with 50 μl of Master mix of the Cobas TaqMan (Roche) and investigated by real-time PCR. It is possible with this procedure to find a reduction in the CT value compared with non-concentrated serum which has been purified using MinElute.

### EXAMPLE 15

### Nucleic Acid Purification with Amino-Modified, Porous Magnetic Polymer Particles

10 μl of a 1 mg/ml solution of the standard plasmid pUC 21 are added to 300 μl of 100 mM ammonium acetate, pH 5.5, and 20 μl of a 100 mg/ml suspension of amino-modified, porous magnetic polymer particles from example 7 or 8 in a 1.5 ml Eppendorff tube. The solution is thoroughly mixed by pulse vortexing for ten seconds, and the DNA attachment is assisted by shaking the tube at 1100 rpm in an Eppendorff thermomixer for 5 minutes. The particles are separated from the supernatant by magnetic removal in a microtube magnetic separator, for example a 12-tube magnetic separator (Qiagen GmbH, Hilden, Germany, cat. No. 36912), and the supernatant is discarded. Then 500 μl of double-distilled water are added, and the tube is pulse-vortexed for ten seconds. The particles are then removed from the supernatant by magnetic removal, and the supernatant is discarded. This washing sequence is repeated and the supernatant is again discarded after the magnetic removal. The particles are then suspended in 50 μl of “TE” buffer (10 mM tris/Cl; pH 8.0, 1 mM EDTA) with 0.1% SDS (sodium dodecylsulfate, Sigma-Aldrich, Fluka cat. No. 71725), and the suspension is thoroughly mixed by pulse vortexing for ten seconds. The tube is then placed on a magnetic separator and the supernatant is transferred into a second Eppendorff tube. After the purification, the DNA content of the eluates can be determined by OD₃₂₀ measurements and by polyacrylamide gel electrophoresis. As an alternative thereto, the DNA can be eluted by a buffer with a high salt content (for example 1.25 ml of NaCl; 50 mM MOPS, pH 8.5, 15% isopropanol), but it must be subsequently precipitated in order to make it usable in subsequent biochemical reactions.

### EXAMPLE 16

### Binding of Primary Antibodies to Porous Magnetic Polymer Particles Modified with Secondary Antibodies

3.4×10⁵/ml human CD3+ and 4.2×10⁵/ml CD3− suspension cells are mixed in a solution containing 10 mM Na₂HPO₄, 100 mM NaCl (pH 7.5) and 10% of a fetal calf serum (FCS). Then 3.32×10⁶ cells are used per experiment. The mixture is incubated with CD3-specific monoclonal antibodies (mouse anti human CD3 PerCP/Becton Dickinson GmbH, Heidelberg, Germany, cat. No. 555330) without shaking for 30 minutes. Subsequently, 1 ml of phosphate-buffered saline (10 mM Na₂HPO₄, 100 mM NaCl/pH 7.5) is added and the mixture is centrifuged at 1000 rpm. The supernatant is removed by pipetting, and the particles are resuspended in 1 ml of phosphate-buffered saline. Then 200 μl of a 25 mg/ml suspension of magnetic particles functionalized with secondary antibodies (goat anti mouse IgG) as in example 11 or 12 are added. The particles are incubated and mixed by occasional shaking for 20 minutes. In the next step, the tube is placed on a magnetic separator, and the supernatant is transferred into a second tube. Subsequently, in a further step, 20 μl of mouse anti-human CD3 PerCP are added and incubated for 15 minutes, washed and analyzed by fluorescence activated cell sorting (FACS). Analysis of the signals showed
a) unstained cells,
b) CD3 PerCP stained and unseparated cell mixtures,
c) cells which were incubated with separating antibody followed by incubation with staining antibody (CD3 PerCP) and
d) magnetically removed cells stained with CD3 Per CP.

The FACS results showed a depletion in CD3-expressing Jurkat cells by at least 85% from 39.3 to 5.44%.

### EXAMPLE 17

### Nucleic Acid Purification with Carboxylate-Modified, Porous Magnetic Polymer Particles (Gel Extraction)

A 200 mg gel fragment containing 1 μg of DNA is put into 400 μl of “QX1” buffer (Qiagen GmbH, Hilden, Germany, cat. No. 20912) and thoroughly mixed by pulse vortexing for five seconds. Then 50 μl of a 50 mg/ml suspension of carboxylate-modified porous magnetic polymer particles according to example 3 or 10 are added, and the mixture is mixed by renewed pulse vortexing. The mixture is then heated on a heating block at 50° C. for five minutes and again mixed by pulse vortexing for 10 seconds. The particles are then removed from the supernatant by magnetic removal and the supernatant is discarded. In the next step, 500 μl of the “QX1” buffer are added, and the suspension is thoroughly mixed by pulse vortexing for five seconds. The removal step is then repeated, and the supernatant is again discarded. This is followed by two washing steps with the “PE” buffer (Qiagen GmbH; Hilden, Germany, cat. No. 19065), and the respective supernatants are discarded. The particles are then dried once by rotating the tubes for 10 minutes without removing the magnetic separator during this. After the drying step, 100 μl of the “EB” buffer (Qiagen GmbH, Hilden, Germany, cat. No. 19068) are added, and the suspension is mixed by pulse vortexing for 15 seconds. The supernatant is removed from the particles by means of a magnetic separator and transferred into another tube. The elution step is repeated, and the eluates are collected and homogenized by brief pulse vortexing. The purity and amount of the purified DNA can be determined by gel electrophoresis and OD measurements.

1. Magnetic polymer particles, characterized in that the magnetic particles are embedded in a polyacrylate or poly[(alkylacrylate)]matrix and wherein the magnetic polymer particles have a maximum pore radius in a range of from 20 to 500 nm.

2. Magnetic polymer particles as claimed in claim 1, characterized in that the particles have a maximum pore radius in a range of from 30 to 400 nm.

3. Magnetic polymer particles as claimed in claim 2, characterized in that the particles have a maximum pore radius in a range of from 80 to 250 nm.

4. Magnetic polymer particles as claimed in claim 1, characterized in that the particles have an average particle size in a range of from 5 to 25 μm.

5. Magnetic polymer particles as claimed in claim 4, characterized in that the particles have an average particle size in a range of from 6 to 20 μm.

6. Magnetic polymer particles as claimed in claim 5, characterized in that the particles have an average particle size in a range of from 10 to 15 μm.

7. Magnetic polymer particles as claimed in claim 1, characterized in that the particles are selected from the_group consisting of ferromagnetic, ferrimagnetic and superparamagnetic particles, and combinations thereof.

8. Magnetic polymer particles as claimed in claim 7, wherein the ferromagnetic and/or ferrimagnetic particles are selected from the group consisting of γ-Fe₂O₃ (maghemite) and Cr₂O₃.

9. Magnetic polymer particles as claimed in claim 8, wherein the ferromagnetic or ferrimagnetic particles are selected from the group of ferrites of the type (M²⁺O)Fe₂O₃, wherein M²⁺ is a divalent transition metal cation.

10. Magnetic polymer particles as claimed in claim 9, characterized in that the magnetic material is Fe₃O₄ (magnetite).

11. Magnetic polymer particles as claimed in claim 1, characterized in that the particles have a crosslinker content of from 1-95 wt %, a functionalized polymer content of from 1-99 wt %, and a magnetic material content of from 1-95 wt %.

12. Magnetic polymer particles as claimed in claim 11, characterized in that the particles have a crosslinker content of from 10-80 wt %, a functionalized polymer content of from 10-80 wt %, and a magnetic material content of from 10-80 wt %.

13. Magnetic polymer particles as claimed in claim 12, characterized in that the particles have a crosslinker content of from 20-70 wt %, a functionalized polymer content of from 20-70 wt %, and a magnetic material content of from 20-70 wt %.

14. Magnetic polymer particles as claimed in claim 13, characterized in that the particles have a crosslinker content of from 15-40 wt %, a functionalized polymer content of from 30-60 wt %, and a magnetic material content of from 30-60 wt %.

15. Magnetic polymer particles as claimed in claim 1, characterized in that the ferromagnetic, ferrimagnetic and/or superparamagnetic particles are embedded in a crosslinked polyacrylate or poly[alkylacrylate]matrix comprising functional groups of the general formula (I)
—C(═O)—M—R (I)
in which wherein
M may be —O—, —NH—or —N(C₁-C₆-alkyl)-;
R may be hydrogen or YX wherein
Y may be an alkylene group —(CH₂)ₗ— and l may be an integer from 1 to 6;
a hydroxy-substituted alkylene group comprising:
—[CH₂—CH(OH)—CH₂]_{g}— and/or —[CH_{2—}CH(CH₂OH)—]ₕ—, and
wherein g and h may be independently of one another an integer from 1 to 6;
—CH₂—CH₂CH(OH)— and/or —CH₂—CH₂—CH(OH)—CH₂—CH₂CH(OH)—
—[CH₂—CH(OH)]ₘ— and/or —[CH(OH)—CH₂]ₙ—, and
wherein m and n may be independently of one another an integer from 1 to 6;
—(CH₂)ₐ—CH(OH)—CH₂-A-(CH2)_{b}-B—C(═O)-[cyclo-C₆H₁₀]—CH₂—,
wherein A and B may be independently of one another —NH—, —N(C₁-C₆-alkyl)- or —O— and a may be an integer from 1 to 6 and b may be an integer from 1 to 8;
X may be hydrogen, —OH, —O—C₁-C₆-alkyl, —O—C₆-C₁₀-aryl, —O—C₇-C₁₄-alkylaryl with an alkylene chain comprising from 1 to 6 carbon atoms and a C₆-C₁₂-aryl radical;
—C₁-C₆-alkyl, —C₆-C₁₂-aryl, heteroaryl, an imidazolyl radical which is optionally linked via a C₁-C₆-alkylene group;
C₇-C₁₄-alkylaryl with an alkylene chain comprising from 1 to 6 carbon atoms and a C₆-C₁₂-aryl radical;
a substituent of the general formula 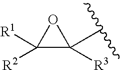
wherein
R¹, R² and R³ may be independently of one another hydrogen, C₁-C₆-alkyl and/or C₆-C₁₀-aryl;
—CN, —NC, —N₃;
—C(═O)—R⁴ and wherein R⁴ may be hydrogen, OH, C₁-C₆-alkyl, —O—C₁-C₆-alkyl, C₆-C₁₀-aryl or —O—C₆-C₁₂-aryl;
—NH₂, —NHR⁵, and wherein R⁵ may be hydrogen, C₁-C₆-alkyl and/or C₆-C₁₀-aryl;
F, Cl, Br or I;
—SH or —S—S—H;
2-thiopyridyl or 4-thiopyridyl;
—S(═O)—CH₂—CF₃;
acylimidazole, maleimido or azlactone groups;
or
R may be Y′X′L wherein
Y′ may be a single bond;
an alkylene group —(CH₂)_{q}—, and wherein q may be an integer from 1 to 6;
a hydroxy-substituted alkylene group comprising:
—[CH₂—CH(OH)—CH₂]ᵢ— and/or —[CH_{2—}CH(CH₂OH)—]ₒ—, and
wherein i and o may be independently of one another an integer from 1 to 6;
—CH₂—CH₂CH(OH)— and/or —CH₂—CH₂—CH(OH)—CH₂—CH₂CH(OH)—
—[CH₂—CH(OH)]ᵣ— and/or —[CH(OH)—CH₂]ₛ—, and
wherein r and s may be independently of one another an integer from 1 to 6;
—(CH₂)ₐ—CH(OH)—CH₂-A-(CH2)_{b}-B—C(═O)-[cyclo-C₆H₁₀]—CH₂—,
where A and B may be independently of one another —NH—, —N(C₁-C₆-alkyl)- or —O— and
a may be an integer from 1 to 6, and b may be an integer from 1 to 8;
—(CH₂)ₐ—CH(OH)—CH₂-A-(CH2)_{b}-B—C(═O)-[cyclo-C₆H₁₀]—CH₂—, and
wherein A and B may be —NH—, a may be an integer from 1 to 2, and b is 6;
X′ may be a single bond;
—CH(OH)—CH₂—O—, —CH(OH)—CH₂—S—, —CH(OH)—CH₂—NH—, —CH(OH)—CH₂—N(C₁-C₆-alkyl)-, —O—, —C(═O)O—, —C(═O)NH—, —C(═O)N(C₁-C₆-alkyl)-;
—CR¹R²—R³CH—O—, —O—CR¹R²—CHR³—, wherein R¹, R² and R³ may be independently of one another hydrogen, C₁-C₆-alkyl or C₆-C₁₀-aryl;
—CN, —NC, —N₃;
—C(═O)—R⁴ wherein R⁴ may be hydrogen, OH, C₁-C₆-alkyl, —O—C₁-C₆-alkyl, C₆-C₁₀-aryl or —O—C₆-C₁₂-aryl;
—NH₂, —NHR⁵ wherein R⁵ may be hydrogen, C₁-C₆-alkyl and/or C₆-C₁₀-aryl;
F, Cl, Br or I;
—SH or —S—S—H;
2-thiopyridyl or 4-thiopyridyl;
—S(═O)—CH₂—CF₃;
acylimidazole, maleimido or azlactone groups;
—NH— or —N(C₁-C₆-alkyl)-;
L may be —C(═O)—NH—(CH₂)ᵤ—[NH—(CH₂)₂]ᵥ—NH₂, and wherein u and v may be independently of one another an integer from 1 to 4;
—(CH₂)_{w}—C(═O)OH and wherein w may be an integer from 1 to 6;
a tri-, tetra- or pentadentate chelating agent selected from the group consisting of a nitrilotriacetic acid residue linked via its ε-N, a low molecular weight, high molecular weight, or linear polyethyleneimine residue having a molecular weight of from 500 to 200,000 Da, an amino radical, polyamine residue, spermidine, cadaverine, diethylenetriamine, spermine, 1,4-bis(3-aminopropyl)piperazine, 1-(2-aminoethyl)piperazine, 1-(2-aminoethyl)piperidine, 1,4,10,13-tetraoxa-7,16-diazacyclooctadecane, a carboxyl acid residue, a bound antibody, a secondary antibody, proteins, biotin, oligonucleotides or streptavidin, IDA, DEO, or TED, and
—CH₂—CH₂—N—(CH₂COO⁻)[CH(COO⁻)CH₂COO⁻)].

16. Magnetic polymer particles as claimed in claim 15, characterized in that
M may be —O—, —NH— or —N(C₁-C₆-alkyl)-;
R may be hydrogen
or
—YX wherein
Y may be an alkylene group —(CH₂)ₗ— and l may be an integer from 1 to 6;
a hydroxy-substituted alkylene group comprising:
—[CH₂—CH(OH)—CH₂]_{g}— and/or —[CH_{2—}CH(CH₂OH)—]ₕ—, and
wherein g and h may be independently of one another an integer from 1 to 4;
—CH₂—CH₂CH(OH)— and/or —CH₂—CH₂—CH(OH)—CH₂—CH₂CH(OH)—,
—[CH₂—CH(OH)]ₘ— and/or —[CH(OH)—CH₂]ₙ—, and
wherein m and n may be independently of one another an integer from 1 to 4;
—(CH₂)ₐ—CH(OH)—CH₂-A-(CH2)_{b}-B—C(═O)-[cyclo-C₆H₁₀]—CH₂—,
wherein A and B may be —NH—, a may be an integer from 1 to 2, and b is 6;
X may be hydrogen, —OH, —O—C₁-C₄-alkyl, —O—C₆-C₁₀-aryl, —O—C₇-C₁₄-alkylaryl with an alkylene chain comprising from 1 to 6 carbon atoms and a C₆-C₁₂-aryl radical;
a substituent of the general formula 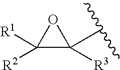
wherein
R¹, R² and R³ may be independently of one another hydrogen, C₁-C₃-alkyl;
—NH₂, —NHR⁵, and wherein R⁵ may be hydrogen, C₁-C₄-alkyl;
F, Cl or Br;
—CN, —NC;
—SH or —S—S—H;
2-thiopyridyl or 4-thiopyridyl;
—S(═O)—CH₂—CF₃ (tresyl);
acylimidazole, maleimido or azlactone groups;
or
R may be —Y′X′L wherein
Y′ may be a single bond;
an alkylene group —(CH₂)_{q}—, and wherein q may be an integer from 1 to 6;
a hydroxy-substituted alkylene group comprising:
—[CH₂—CH(OH)—CH₂]ᵣ— and/or —[CH_{2—}CH(CH₂OH)—]ₒ—, and
wherein i and o may be independently of one another an integer from 1 to 4;
—CH₂—CH₂CH(OH)— and/or —CH₂—CH₂—CH(OH)—CH₂—CH₂CH(OH)—;
—[CH₂—CH(OH)]ᵣ— and/or —[CH(OH)—CH₂]ₛ—, and
wherein r and s may be independently of one another an integer from 1 to 4;
—(CH₂)ₐ—CH(OH)—CH₂-A-(CH2)_{b}-B—C(═O)-[cyclo-C₆H₁₀]—CH₂—,
in which A and B may be —NH—, a may be an integer from 1 to 2, and b is 6;
X′ may be a single bond,
—CR¹R²—R³CH—O—, —O—CR¹R²—CHR³— in which R¹, R² and R³ may be independently of one another hydrogen, C₁-C₃-alkyl;
—NH₂, —NHR⁵ and R⁵ may be hydrogen, C₁-C₄-alkyl;
F, Cl or Br;
—CN, —NC;
—SH or —S—S—H;
2-thiopyridyl or 4-thiopyridyl;
—S(′O)—CH₂—CF₃ (tresyl);
acylimidazole, maleimido or azlactone groups;
—CH(OH)—CH₂—O—, —CH(OH)—CH₂—S—, —CH(OH)—CH₂—NH—, —CH(OH)—CH₂—, —N(C₁-C₃-alkyl)-, —O—, —C(═O)O—, —C(═O)NH—, —C(═O)N(C₁-C₃-alkyl)-;
—CR¹R²—R³CH—O−, —O—CR¹R²—CHR³— in which R¹, R² and R³ may be independently of one another hydrogen, C₁-C₃-alkyl;
—NH₂, —NHR⁵ and R⁵ may be hydrogen, C₁-C₄-alkyl;
F, Cl or Br;
—CN, —NC;
—SH or —S—S—H;
2-thiopyridyl or 4-thiopyridyl;
—S(═O)—CH₂—CF₃ (tresyl);
acylimidazole, maleimido or azlactone groups;
—NH— or —N(C₁-C₃-alkyl)-;
L may be —C(═O)—NH—(CH₂)ᵤ—[NH—(CH₂)₂]ᵥ—NH₂, and wherein u and v may be independently of one another in each case an integer from 1 to 4;
—(CH₂)_{w}—C(═O)OH, and wherein w may be an integer from 1 to 4;
a tri-, tetra- or pentadentate chelating agent selected from the group consisting of a nitrilotriacetic acid residue linked via its ε-N, a low molecular weight, high molecular weight., or linear polyethyleneimine residue having a molecular weight of from 500 to 200,000 Da, a polyamine residue, spermidine, cadaverine, diethylenetriamine, spermine, 1,4-bis(3-aminopropyl)piperazine, 1-(2-aminoethyl)piperazine, 1-(2-aminoethyl)piperidine, 1,4,10,13-tetraoxa-7,16-diazacyclooctadecane, a carboxyl acid residue, a bound antibody, a secondary antibody, proteins, biotin, oligonucleotides, streptavidin, IDA, DEO, TED, and
—CH₂—CH₂—N—(CH₂COO⁻)[CH(COO⁻)CH₂COO⁻)].

17. Magnetic polymer particles as claimed in claim 16, characterized in that
M may be —O— or —NH—;
R may be hydrogen
or
YX wherein
Y may be a single bond;
an alkylene group —(CH₂)ₗ—, and wherein l may be an integer from 1 to 6;
a hydroxy-substituted alkylene group:
—[CH₂—CH(OH)—CH₂]_{g}— and/or —[CH_{2—}CH(CH₂OH)—]ₕ—, and
wherein g and h may be independently of one another an integer from 1 to 2;
CH₂—CH₂CH(OH)— and/or —CH₂—CH₂—CH(OH)—CH₂—CH₂CH(OH)—;
—[CH₂—CH(OH)]ₘ— and/or —[CH(OH)—CH₂]ₙ—, and
wherein m and n may be independently of one another an integer 1 or 2,
X may be hydrogen, —OH, —O—C₁-C₄-alkyl, —O—C₆-C₁₀-aryl;
a substituent of the general formula: 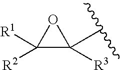
wherein R¹, R² and R³ may be independently of one another hydrogen or C₁-C₂-alkyl;
—NH₂;
Cl or Br;
—S(═O)—CH₂—CF₃;
or
R may be Y′X′L wherein
Y′ may be a single bond;
an alkylene group —(CH₂)_{q}— and wherein q may be an integer from 1 to 3;
a hydroxy-substituted alkylene group comprising —[CH₂—CH(OH)—CH₂]ᵢ— and —[CH_{2—}CH(CH₂OH)—]ₒ—, and
wherein i and o may be independently of one another an integer from 1 to 2;
—[CH₂—CH₂CH(OH)]— or
[CH₂—CH(OH)]ᵣ— and/or —[CH(OH)—CH₂]ₛ—, and
wherein r and s may be independently of one another an integer from 1 2;
X′ may be a single bond;
—CR¹R²—R³CH—O—, —O—CR¹R²—CHR³— in which R¹, R² and R³ may be independently of one another hydrogen or C₁-C₂-alkyl;
—NH₂;
Cl or Br;
—S(═O)—CH₂—CF₃;
—CH(OH)—CH₂—O—, —CH(OH)—CH₂—S—, —CH(OH)—CH₂—NH—, —CH(OH)—CH₂—N(C₁-C₃-alkyl)-, —O—, —C(═O)O—, —C(═O)NH—, —C(═O)N(C₁-C₃-alkyl)-;
—NH—;
L may be
a tri-, tetra- or pentadentate chelating agent selected from the group consisting of a nitrilotriacetic acid residue linked via its ε-N, a low molecular weight, high molecular weight., or linear polyethyleneimine residue having a molecular weight of from 500 to 200,000 Da, spermidine, cadaverine, diethylenetriamine, spermine, 1,4-bis(3-aminopropyl)piperazine, 1-(2-aminoethyl)piperazine, 1-(2-aminoethyl)piperidine, 1,4,10,13-tetraoxa-7,16-diazacyclooctadecane, a carboxyl acid residue, a bound antibody, a secondary antibody, proteins, biotin, oligonucleotides and streptavidin;
—C(═O)—NH—(CH₂)₂—[NH—(CH₂)ᵤ]—NH₂, and wherein u may be 2 and v may be 2;
—(CH₂)_{w}—C(═O)OH and w may be an integer from 1 to 2;
NTA, IDA, TED, or
—CH₂—CH₂—N—(CH₂COO⁻)[CH(COO⁻)CH₂COO⁻)].

18. Magnetic polymer particles as claimed in claim 17, characterized in that
M may be —O— or —NH—;
R may be hydrogen or
YX wherein
Y may be an alkylene group —(CH₂)ₗ—, and wherein l may be an integer from 1 to 6;
a hydroxy-substituted alkylene group comprising:
—[CH₂—CH(OH)—CH₂]_{g}— and/or —[CH_{2—}CH(CH₂OH)—]ₕ—, and
wherein g and h may be independently of one another an integer from 1 to 2;
—CH₂—CH₂CH(OH)—;
—[CH₂—CH(OH)]ₘ— and/or —[CH(OH)—CH₂]ₙ—, and
wherein m and n may be independently of one another an integer from 1 to 2;
X may be hydrogen;
a substituent of the general formula 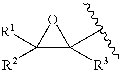
wherein
R¹, R² and R³ may be hydrogen;
—NH₂,
or
Y′X′L wherein
Y′ may be a single bond;
an alkylene group —(CH₂)_{q}—, and wherein q may be an integer from 1 to 6;
a hydroxy-substituted alkylene group comprising:
—[CH₂—CH(OH)—CH₂]ᵣ— and/or —[CH_{2—}CH(CH₂OH)—]ₒ—, and
wherein i and o may be independently of one another an integer from 1 to 2;
—CH₂—CH₂CH(OH)— and
—[CH₂—CH(OH)]ᵣ— and —[CH(OH)—CH₂]ₛ—
where r and s may be independently of one another an integer from 1 to 2;
X′ may be a single bond,
—CH(OH)—CH₂—O—, —CH₂—CH(OH)—O—,
—CR¹R²—R³CH—O—, —O—CR¹R²—CHR³— in which R¹, R² and R³ above may be hydrogen;
—NH—;
L may be a tri-, tetra- or pentadentate chelating agent selected from the group consisting of a nitrilotriacetic acid residue, a polyethyleneimine residue, an amino radical, a polyamine residue, a carboxyl acid residue, a bound antibody, proteins, biotin, oligonucleotides, spermine, streptavidin, a secondary antibody;
—C(═O)—NH—(CH₂)₂—[NH—(CH₂)ᵤ]ᵥ—NH₂, and wherein u may be 1 or 2, and v may be 2;
—(CH₂)_{w}—C(═O)OH and w may be an integer from 1 to 2;
NTA, IDA/DEO, TED, or
—CH₂—CH₂—N—(CH₂COO⁻)[CH(COO⁻)CH₂COO⁻)].

19. Magnetic polymer particles as claimed in claim 1, wherein the polymer matrix is crosslinked with an acrylate crosslinker selected from the group consisting of a di- or polyacrylate or a di- or polyalkylacrylate.

20. Magnetic polymer particles as claimed in claim 19, where the crosslinker is selected from the group consisting of ethylene glycol acrylates, ethylene glycol (alkyl)acrylates, ethylene glycol methacrylates, polyethylene glycol acrylates, polyethylene glycol (alkyl)acrylates, polyethylene glycol methacrylates, ethylene glycol acrylates, ethylene glycol (alkyl)acrylates, ethylene glycol methacrylates, polyethylene glycol acrylates, polyethylene glycol (alkyl)acrylates, in particular polyethylene glycol methacrylates, pentaerythritol tetraacrylates, pentaerythritol triacrylates, propylene glycol acrylates, propylene glycol (alkyl)acrylates, propylene glycol methacrylates, polypropylene glycol acrylates, polypropylene glycol (alkyl)acrylates, in particular polypropylene glycol methacrylates propylene glycol acrylates, propylene glycol (alkyl)acrylates, propylene glycol methacrylates, polypropylene glycol acrylates, polypropylene glycol (alkyl)acrylates, and polypropylene glycol methacrylates.

21. A method for preparing magnetic polymer particles comprising the steps:
a) preparing a dispersion of magnetic particles which are selected from the group consisting of ferromagnetic, ferrimagnetic and superparamagnetic particles in a first organic phase, where the first organic phase comprises:
a.1.) one or more acrylate monomer(s) selected from the group consisting of acrylic acid, methacrylic acid, acrylates, and methacrylates, having substituted carboxyl groups comprising —C(═O)—O—Y—X, wherein Y is a spacer group and X is a reactive group,
a.2.) at least one crosslinker comprising two or more acrylate or (alkyl)acrylate groups,
a.3.) at least one lipophilic free-radical initiator, and
a.4.) at least one organic pore former;
b) homogenizing the dispersion and a second organic phase to form an emulsion, where the second organic phase comprises:
b1) at least one liquid hydrophobic compound and
b2) at least one surface-active substance, and
c) free-radical polymerizing the emulsion,
wherein the magnetic polymer particles have an average particle size in the range of from 5 μm to 25 μm and a maximum pore radius in the range of from 20 to 500 nm.

22. The method as claimed in claim 21, wherein the emulsion is flushed with an inert gas before the free-radical polymerization, and the free-radical polymerization is carried out in an inert gas atmosphere.

23. The method as claimed in claim 21, wherein the magnetic particles are ground and/or deagglomerated before or during preparation of the dispersion.

24. The method as claimed in claim 21, wherein the free-radical polymerization is carried out at a temperature between 50° C. and 120° C.

25. The method as claimed in a claim 24, wherein the free-radical polymerization is carried out at a temperature between 60° C. and 90° C.

26. The method as claimed in claim 21, wherein the monomer in the first organic phase is a compound according to the general formula II:
H₂C═CR′—C(═O)—OR (II)
where R′ is H— or a C₁-C₃-alkyl,
and
R is hydrogen or —Y—X according to any of claims 15 to
Y may be an alkylene group —(CH₂)ₗ— and l may be an integer from 1 to 6;
a hydroxy-substituted alkylene group comprising:
—[CH₂—CH(OH)—CH₂]_{g}— and/or —[CH_{2—}CH(CH₂OH)—]ₕ—, and
wherein g and h may be independently of one another an integer from 1 to 6;
—CH₂—CH₂CH(OH)— and/or —CH₂—CH₂—CH(OH)—CH₂—CH₂CH(OH)—
—[CH₂—CH(OH)]ₘ— and/or —[CH(OH)—CH₂]ₙ, and
wherein m and n may be independently of one another an integer from 1 to 6;
—(CH₂)ₐ—CH(OH)—CH₂-A-(CH2)_{b}-B—C(═O)—[cyclo-C₆H₁₀]—CH₂—,
wherein A and B may be independently of one another —NH—, —N(C₁-C₆-alkyl)- or —O—and a may be an integer from 1 to 6 and b may be an integer from 1 to 8;
X may be hydrogen, —OH, —O—C₁-C₆-alkyl, —O—C₆-C₁₀-aryl, —O—C₇-C₁₄-alkylaryl with an alkylene chain comprising from 1 to 6 carbon atoms and a C₆-C₁₂-aryl radical;
—C₁-C₆-alkyl, —C₆-C₁₂-aryl, heteroaryl, an imidazolyl radical which is optionally linked via a C₁-C₆-alkylene group;
C₇-C₁₄-alkylaryl with an alkylene chain comprising from 1 to 6 carbon atoms and a C₆-C₁₂-aryl radical;
a substituent of the general formula 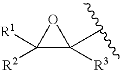
wherein
R¹, R² and R³ may be independently of one another hydrogen, C₁-C₆-alkyl and/or C₆-C₁₀-aryl;
—CN, —NC, —N₃;
—C(═O)—R⁴, and wherein R⁴ may be hydrogen, OH, C₁-C₆-alkyl, —O—C₁-C₆-alkyl, C₆-C₁₀-aryl or —O—C₆-C₁₂-aryl;
—NH₂, —NHR⁵, and wherein R⁵ may be hydrogen, C₁-C₆-alkyl and/or C₆-C₁₀-aryl;
F, Cl, Br or I;
—SH or —S—S—H;
2-thiopyridyl or 4-thiopyridyl;
—S(═O)—CH₂—CF₃;
acylimidazole, maleimido or azlactone groups.

27. The method as claimed in claim 26, wherein the spacer Y is a —(CH₂)ₗ— group and l is an integer from 1 to 6.

28. The method as claimed in claim 21, wherein the monomer in the first organic phase is selected from the group consisting of glycidyl methacrylate, 2-hydroxyethyl methacrylate, methacrylic acid, acrylic acid, and acrylic acid derivatives of the general formula (III)
H₂C═CR′C(═O)O—(CH₂)_{c}Z (III)
wherein
R¹ is H or methyl;
c is an integer from 1 to 6;
Z is selected from the group consisting of —OH, —NH₂, —C(═O)OH, halogen, tresyl, maleimido, and epoxy groups.

29. The method as claimed in claim 21, wherein the crosslinker is at least one alkylidene glycol diacrylate or alkylidene glycol (alkyl)acrylate of the general formula (IV):
H₂C═CR″C(═O)O—[(C_{d}H_{2d}O)]ₑ(C═O)CR′″═CH₂ (IV)
wherein R″ and R′″ are independently of one another H or a C₁-C₃-alkyl, and R″ and R′″ are both H or methyl, d is an integer from 1 to 4 and e is an integer from 1 to 100.

30. The method as claimed in claim 29, characterized in that d is an integer from 1 to 2 and e is an integer from 1 to 4.

31. The method as claimed in claim 21, characterized in that the crosslinker is ethylene glycol diacrylate, ethylene glycol dimethacrylate, or a mixture thereof.

32. The method as claimed in claim 21, characterized in that the crosslinker is a polyacrylate or polymethacrylate having at least two acrylic or methacrylic groups.

33. The method as claimed in claim 21, characterized in that the crosslinker is a polyacrylate or a polymethacrylate having 3 or 4 acrylic or methacrylic groups.

34. The method as claimed in claims 21, characterized in that the crosslinker is selected from the group consisting of pentaerythritol tetraacrylate, pentaerythritol tetramethacrylate, pentaerythritol triacrylate, and pentaerythritol trimethacrylate, or mixtures thereof.

35. The method as claimed in claim 21, wherein the organic pore former is selected from the group consisting of:
a) aliphatic, branched or unbranched alcohols having from 4 to 20 carbon atoms, with one or more hydroxy groups,
b) alkylidene glycols, and
c) polymeric compounds having a mass-average molecular mass M_{w} between 200 and 100,000 g/mol and wherein the compounds are selected from the group consisting of polyalkylidene glycol derivatives, polyethyleneimine, polyvinylpyrollidone, and polystyrene.

36. The method as claimed in claim 21, wherein the lipophilic free-radical initiator is selected from the group consisting of azoisobutyronitrile (AIBN), 2,2′-azobis(2-amidinopropane)dihydrochloride, 2,2′-azobis(2,4-dimethylvaleronitrile), and 1,1′-azobis(cyclohexane-1-carbonitrile).

37. The method as claimed in claim 21, wherein the hydrophobic liquid is selected from the group consisting of aliphatic alkanes and cyclic alkanes, wherein the aliphatic alkanes have the general formula C₂H₂ₙ₊₂, wherein n is greater than 6, and aliphatic alkenes and cyclic alkenes, wherein the aliphatic alkenes have the general formula C₂H₂ₙ, wherein n is greater than 6.

38. The method as claimed in claim 21, wherein the hydrophobic liquid is an aromatic or heteroaromatic compound which may be substituted with alkyl or alkene groups.

39. The method as claimed in claim 38, wherein the hydrophobic liquid is toluene.

40. The method as claimed in claim 21, wherein the surface-active substance is an emulsifier selected from the group consisting of cationic, anionic, and nonionic emulsifiers.

41. The method as claimed in claim 21, where the magnetic particles are ferromagnetic and/or ferrimagnetic particles.

42. The method as claimed in claim 41, characterized in that the magnetic particles are selected from the group consisting of γ-Fe₂O₃ (maghemite), Cr₂O₃, and ferrites.

43. The method as claimed in claim 42, characterized in that the ferrite is (M²⁺O)Fe₂O₃, and wherein the M²⁺ is a divalent transition metal cation.

44. The method as claimed in claim 42, characterized in that the magnetic particles are Fe₃O₄ (magnetite).

45. The method as claimed in claim 21, characterized in that the crosslinker is present in a ratio of 0.1-20 wt %.

46. The method as claimed in claim 45, characterized in that the crosslinker is present in a ratio of from 0.5 to 5 wt %.

47. The method as claimed in claim 46, characterized in that the crosslinker is present in a ratio of from 1 to 4 wt %.

48. The method as claimed in claim 21, characterized in that the functionalized monomer is present in a ratio of from 0.1 to 20 wt %.

49. The method as claimed in claim 48, characterized in that the functionalized monomer is present in a ratio of from 0.5 to 5 wt %.

50. The method as claimed in claim 49, characterized in that the functionalized monomer is present in a ratio of from 1 to 4 wt %.

51. The method as claimed in claim 21, characterized in that the magnetic material is present in a ratio of from 0.1 to 20 wt %.

52. The method as claimed in claim 51, characterized in that the magnetic material is present in a ratio of from 0.5 to 5 wt %.

53. The method as claimed in claim 52, characterized in that the magnetic material is present in a ratio of from 1 to 4 wt %.

54. The method as claimed in claims 21 to 53, characterized in that the initiator is present in a ratio of from 0 to 5 wt %.

55. The method as claimed in claim 54, characterized in that the initiator is present in a ratio of from 0.01 to 3 wt %.

56. The method as claimed in claim 55, characterized in that the initiator is present in a ratio of from 0.05 to 0.5 wt %.

57. The method as claimed in claim 21, characterized in that the detergent is present in a ratio of from 0 to 20 wt %.

58. The method as claimed in claim 57, characterized in that the detergent is present in a ratio of from 0.1 to 10 wt %.

59. The method as claimed in claim 58, characterized in that the detergent is present in a ratio of from 0.1 to 3 wt %.

60. The method as claimed in claim 21, characterized in that the organic pore former is present in a ratio of from 0.1 to 20 wt %.

61. The method as claimed in claim 60, characterized in that the organic pore former is present in a ratio of from 0.5 to 5 wt %.

62. The method as claimed in claim 61, characterized in that the organic pore former is present in a ratio of from 1 to 4 wt %.

63. The method as claimed in claim 21, further comprising step d) functionalizing the magnetic polymer particles by linking a ligand which immobilizes biomolecules to the magnetic polymer.

64. The method as claimed in claim 63, where the ligand is linked by, in a first step d1), covalently bonding a spacer compound having at least two reactive groups to the carboxyl groups of the magnetic polymer particles and then, in a second step d2), covalently linking the ligand to the magnetic polymer particles.

65. A method for isolating and/or analyzing at least one biomolecular species from a biomolecule-containing sample, where the method comprises the following steps:
a) providing a sample containing at least one biomolecular species,
b) contacting the sample with magnetic polymer particles as claimed in claim 1 under conditions with which the at least one biomolecular species binds to the magnetic polymer particles,
c) removing the magnetic polymer particles with the bound biomolecules by use of at least one magnetic field.

66. The method as claimed in claim 65, further comprising the step:
d) eluting the at least one biomolecular species from the magnetic polymer particles.

67. The method as claimed in claim 65, wherein the at least one biomolecular species is selected from the group consisting of nucleic acids, oligonucleotides, proteins, polypeptides, peptides, carbohydrates, and lipids.

68. The method as claimed in claim 65, wherein the sample containing at least one biomolecular species is selected from the group consisting of blood, tissue, cells, vegetable materials, amplification solutions, and PCR solutions.

69. The method according to claim 21 wherein the magnetic polymer particles have an average particle size in the range of from 6 to 20 μm, and a maximum pore radius in the range from 30 to 400 nm.

70. The method according to claim 21, wherein the magnetic polymer particles have an average particle size in the range of from 10 to 15 μm, and a maximum pore radius in the range of from 80 to 250 nm.

71. The method according to claim 65, wherein the biomolecular species are selected from the group consisting of plasmid DNA, genomic DNA, cDNA, PCR DNA, linear DNA, RNA, ribozymes, aptamers, chemically synthesized or modified nucleic acids, and oligonucleotides, and combinations thereof.
